SEQUENCE LISTING (30 sequences)

SEQ ID NO: 1 - DNA, 24 bp, Artificial sequence
  VH6-1 forward primer
agtgtcagggagatgccgtattca

SEQ ID NO: 2 - DNA, 23 bp, Artificial sequence
  VH6-1 reverse primer
acttcccctcactgtgtctcttg

SEQ ID NO: 3 - DNA, 21 bp, Artificial sequence
  D1-26 forward primer
gggcgcctgggtggattctga

SEQ ID NO: 4 - DNA, 24 bp, Artificial sequence
  D1-26 reverse primer
gtggcccctaaacctgagtctgct

SEQ ID NO: 5 - DNA, 21 bp, Artificial sequence
  D1-20 forward primer
cccgagcaccgtccccattga

SEQ ID NO: 6 - DNA, 24 bp, Artificial sequence
  D1-20 reverse primer
gtgccggtgatccctgtctttctg

SEQ ID NO: 7 - DNA, 22 bp, Artificial sequence
  C-mu1 forward primer
gcgggagtcggccaccatcacg

SEQ ID NO: 8 - DNA, 22 bp, Artificial sequence
  C-mu1 reverse primer
agcacagccgccgccccagtag

SEQ ID NO: 9 - DNA, 20 bp, Artificial sequence
  C-delta1 forward primer
tggggagaggagagcacagt

SEQ ID NO: 10 - DNA, 19 bp, Artificial sequence
  C-delta1 reverse primer
ggcgggcgtaggggtcagc

SEQ ID NO: 11 - DNA, 31 bp, Gallus gallus domesticus
  misc_feature (1)..(31): Immunoglobulin heavy chain constant region
ctcggatcccaacaaacggcactcgataatt

SEQ ID NO: 12 - DNA, 31 bp, Gallus gallus domesticus
  misc_feature (1)..(31): Immunoglobulin heavy chain constant region
ctcgaattcttcattgaccttcattaaccgc

SEQ ID NO: 13 - DNA, 31 bp, Gallus gallus domesticus
  misc_feature (1)..(31): Immunoglobulin light chain variable region
ctcgttaacgatgttgtactgagggatgtgg

SEQ ID NO: 14 - DNA, 31 bp, Gallus gallus domesticus
  misc_feature (1)..(31): Immunoglobulin light chain variable region
ctcgttaaccggtgaacaaggatgttcagta

SEQ ID NO: 15 - DNA, 23 bp, Homo sapiens
  misc_feature (1)..(23): Immunoglobulin heavy chain variable region; 3 - 7 forward primer
ggctgagctgggttttccttgtt

SEQ ID NO: 16 - DNA, 19 bp, Homo sapiens
  misc_feature (1)..(19): Immunoglobulin heavy chain variable region; 3 - 7 reverse primer
ctgtcgccccctggtggtc

SEQ ID NO: 17 - DNA, 24 bp, Homo sapiens
  misc_feature (1)..(24): Immunglobulin heavy chain variable region; 4 - 4 forward primer
cctgcacaagaacatgaaacacct

SEQ ID NO: 18 - DNA, 19 bp, Homo sapiens
  misc_feature (1)..(19): Immunoglobulin heavy chain variable region; 4 - 4 reverse primer
gacccggcctcttgctctg

SEQ ID NO: 19 - DNA, 24 bp, Homo sapiens
  misc_feature (1)..(24): Immunoglobulin heavy chain constant region; delta-gamma 3 interval; forward primer
gctgttggcctttattttctattg

SEQ ID NO: 20 - DNA, 22 bp, Homo sapiens
  misc_feature (1)..(22): Immunoglobulin heavy chain constant region; delta - gamma 3 interval; reverse primer
atttgcaccatttcctgagttg

SEQ ID NO: 21 - DNA, 24 bp, Homo sapiens
  misc_feature (1)..(24): Immunoglobulin heavy chain constant region; delta - gamma 3 interval; forward primer
gtgggtgatagaatttggtgtttg

SEQ ID NO: 22 - DNA, 22 bp, Homo sapiens
  misc_feature (1)..(22): Immunoglobulin heavy chain constant region; delta - gamma 3 interval; reverse primer
gtggtgggcaggatgggatgat

SEQ ID NO: 23 - DNA, 24 bp, Homo sapiens
  misc_feature (1)..(24): Immunoglobulin light chain variable region; forward primer
atgccagggactctaacaaacttc

SEQ ID NO: 24 - DNA, 22 bp, Homo sapiens
  misc_feature (1)..(22): Immunoglobulin light chain variable region; reverse primer
ttcccctcaacaaaaacctctc

SEQ ID NO: 25 - DNA, 19 bp, Homo sapiens
  misc_feature (1)..(19): Immunoglobulin light chain constant region; forward primer
agctcgcccgtcacaaaga

SEQ ID NO: 26 - DNA, 23 bp, Homo sapiens
  misc_feature (1)..(23): Immunoglobulin light chain variable region; reverse primer
aggggaaaacaaggaagcaagtc

SEQ ID NO: 27 - DNA, 20 bp, Homo sapiens
  misc_feature (1)..(20): Immunoglobulin light chain variable region; forward primer
gagagggccaccatcaactg

SEQ ID NO: 28 - DNA, 24 bp, Homo sapiens
  misc_feature (1)..(24): Immunoglobulin light chain variable region; reverse primer
aaccctccaacgaataaatcaaga

SEQ ID NO: 29 - DNA, 23 bp, Homo sapiens
  misc_feature (1)..(23): Immunoglobulin light chain variable region; forward primer
aagtcccctgcatatccacaaaa

SEQ ID NO: 30 - DNA, 21 bp, Homo sapiens
  misc_feature (1)..(21): Immunoglobulin light chain variable region; reverse primer
gctgaggcaatcccactgaga

(19) United States
(12) Patent Application Publication
(10) Pub. No.: US 2014/0155580 A1
(43) Pub. Date: Jun. 5, 2014

(54) TRANSGENIC AVES PRODUCING HUMAN POLYCLONAL ANTIBODIES

(71) Applicant: Synageva BioPharma Corp., Lexington, MA (US)

(72) Inventors: Robert J. Etches, San Mateo, CA (US); Robert M. Kay, San Francisco, CA (US); Philip Leighton, San Francisco, CA (US); Lei Zhu, Palo Alto, CA (US)

(21) Appl. No.: 13/930,973

(22) Filed: Jun. 28, 2013

Related U.S. Application Data
(63) Continuation of application No. 12/835,721, filed on Jul. 13, 2010, now abandoned.
Application No. 12/835,721 is a continuation of application No. 10/508,808, filed on Sep. 22, 2004, now abandoned.

(51) Int. Cl. C07K 16/46

(52) U.S. Cl. CPC C07K 16/461
USPC 530/387.3

(57) ABSTRACT

Human antibodies are produced in transgenic chickens having functional genetic components of the human immune system stably integrated in the genome. Techniques are described to integrate unrearranged human heavy and light chain immunoglobulin loci into the genome of the transgenic chicken.

The functional description of the endogenous immunoglobulin loci yields an animal whose antibody repertoire in response to antigen is fully human.

In the preferred embodiment, the human immunoglobulin locus is at least as large as the native human locus and exhibits functional class switching to yield IgG isotype antibodies. In addition to monoclonal antibodies secreted from immortalized B cell populations, polyclonal antibodies may be obtained from eggs laid by the transgenic chickens of the invention.

### CROSS-REFERENCE TO RELATED APPLICATIONS

This application is a continuation of U.S. Ser. No. 10/508,808 filed Sep. 22, 2004, which application was a 371 of PCT/US03/09288 filed Mar. 24, 2003, which claims priority to U.S. Ser. No. 10/104,057 filed Mar. 22, 2002, which applications are fully incorporated herein by reference.

### FIELD OF THE INVENTION

This invention relates to the field of transgenic animals that produce non-endogenous proteins, particularly human polyclonal antibodies, methods and constructs for genetic engineering to make such animals, related modified cell lines and techniques and non-endogenous proteins derived from such animals.

### BACKGROUND OF THE INVENTION

Antibodies are valuable as therapeutic compounds because of their unique ability to specifically bind to an antigen and are readily produced in animals. However, the human immune system is sensitive enough to detect animal-derived antibodies as foreign substances and react by mounting an immune response. The destruction or neutralization of the therapeutic antibody, together with the immune response itself, reduces the therapeutic utility of many animal-derived antibodies and almost completely prevents their use in the treatment of most human disease.

To develop animal-derived antibodies for human therapy, animals have been genetically modified to produce antibodies that mimic the antibodies made by the human immune system. These transgenic animals contain the functional components of the human immune system in their genomic DNA and respond to antigenic challenge with antibodies that are substantially human because they are encoded by human DNA. However, prior transgenic approaches have certain drawbacks and are only capable of producing monoclonal antibodies using hybridomas.

Presently, substantially human antibodies are produced in mice lacking functional endogenous immunoglobulin genes and having inserted portions of the human immunoglobulin genes (See Lonberg & Kay, U.S. Pat. No. 5,874,299). Significantly, these mice rearrange human genes and exhibit both somatic mutation and class switching wherein to produce isotype G antibodies. Other murine systems offer larger repertoires of human variable regions (See Kucherlapati et al. U.S. Pat. No. 6,139,835) but still rely on a human heavy gene that is smaller than the native human gene. Also, because the knockout in the murine models leaves the constant region of the murine immunoglobulin locus attached, even though a human heavy chain immunoglobulin locus is available in the mass genomic DNA, the possibility for trans-chromosomal switching occurs wherein a human variable region is coupled with a murine constant region to yield a chimeric antibody that is not desirable for human therapeutic use. Also, as noted above, the murine system requires hybridomas and cannot yield polyclonal antibodies.

In many therapeutic applications, polyclonal antibodies are more effective than a monoclonal antibody. A complex antigen can be bound by polyclonal antibodies which can participate in a number of effector functions in a natural immune response. The mixture of antibodies in a polyclonal response may also reduce antigenicity conferred by a monoclonal antibody.

Avian biological systems offer many advantages including efficient farm cultivation, rapid growth, and economical production. Globally, chickens and turkeys are a major source of protein in the human diet. Further, the avian egg offers an ideal biological design, both for massive synthesis of a few proteins and ease of isolation of protein product. However, application of the full range of mammalian transgenic techniques to avian species has been unsuccessful. Most notably, as mentioned previously, the transmission to a mature, living animal of a genetic modification introduced into an avian embryonic stem cell has not been demonstrated.

Genetically engineered ayes offer certain advantages over mammals in general and genetically engineered mice in particular. In addition to the production of polyclonal antibodies that can be isolated from an avian egg in commercially valuable quantities, chickens are phylogenetically more distant from humans and the immune system of ayes reacts more strongly to some human antigens than does the immune system of a mammal. However, because the genetic makeup of ayes is significantly different from the genetic makeup of a human, mouse, or other mammal, unique difficulties arise in creating a transgenic animal capable of responding to antigenic challenge to produce human antibodies. For example, direct injection has significant technical difficulties and conventional technologies have not yet yielded a transgenic chicken that expresses a foreign protein.

The use of retroviral vectors has yielded transgenic chickens of limited utility, however, the use of retroviral vectors cannot accommodate site-specific changes to the avian genome. In addition, the capacity of retroviral vectors to deliver foreign DNA into the genome is limited to about 12.5 kb (Love et al.) whereas many transgenic applications require larger sequences. Specifically, to produce developmentally regulated and tissue specific expression of a human immunoglobulin, from unrearranged human immunoglobulin loci would require on the order of 25-50 kb of human DNA to be incorporated into the chicken genome.

Furthermore, avian transgenesis to yield human antibodies requires new and specifically engineered genetic constructs that properly assemble the immunoglobulin genes necessary to express human antibodies. These modifications, together with a functional disruption of the endogenous avian immune system, require long-term cultures of avian embryonic stem (ES) cells that can be genetically modified by gene targeting or other techniques to modify the endogenous genome, to accept large amounts of foreign DNA, and to contribute to the somatic tissues and the germline of recipient embryos. To date, significant limitations on avian ES cell culturing technology have prevented the establishment of long term ES cell cultures that can be used for transgenesis as described herein.

Thus, the ability to make transgenic birds from ES cells that carry precise genetic modifications in the germline, and to incorporate these cells into somatic tissues and the germline of chimeras, requires technology in both cell culturing and genetic engineering. Although both somatic and germline chimeras have been made using chicken ES cells (cES) (Pain et al. 1996) and using undifferentiated, uncommitted cells in stage X embryos (Petitte et al. 1991), cES cells have not been maintained in a pluripotential state such that gene targeting or other genetic modifications can be demonstrated.

Embryonic stem cell lines suitable for use in transgenesis must be stable and maintain pluripotency when the ES cell is transfected with a genetic construct, when the genetic construct is expressed in the ES cell to allow selection of successfully transformed cells, and during the injection into recipient embryos and the formation of resulting chimeras. Moreover, the somatic tissue of the chimera must exhibit the genetic modifications derived from the embryonic stem cells, and the genetic modification must be identified in the tissue of the chimeric animal. Ideally, the embryonic stem cell contains a transgene that is incorporated into the somatic tissue of a chimeric animal, but is also effectively expressed in a wide variety of tissues in the animal, specifically individual tissue types in which the transgene is designed to be expressed. For example, transgenes encoding DNA derived from the lymphoid elements of the immune system might be targeted to be expressed in B lymphocytes of a chimeric or transgenic animal. In such circumstances, the embryonic stem cell culture must allow transformation of the genome of the ES cell with a transgene containing DNA encoding an exogenous protein, and the ES cell must contribute significantly to the genome of the resulting animal.

To conduct avian transgenesis of a type that has been conducted in mice requires the development of long-term, pluripotent avian ES cells. In many cases, transgenesis techniques necessary to modify ES cells, screen modified ES cells, and manipulate the ES cells for injection into embryos to produce transgenic chickens, requires at least several weeks for all of the steps to be performed. For the ES cells to be useful in transgenesis, the pluripotential state must be maintained for the entire time period up until injection into an embryo and the ES cell must be incorporated into a recipient embryo to a substantial degree to be detected in a resulting chimeric animal.

Unless the ES cell culture conditions are ideal, the cells quickly begin to differentiate and cannot contribute to the somatic tissue of a chimera. Thus, once differentiation occurs, cells in culture are no longer useful. Using current avian ES cell culture techniques, ES cell pluripotency degrades too quickly for the cells to be used to create transgenic or chimeric avians.

Homologous recombination is a technique that introduces genetic modifications in the genome of an animal. The principles of homologous recombination in mouse ES cells were developed in the 1970s. In 1981, two groups derived pluripotent embryonic stem cell lines from mouse blastocysts. Bradley et al. (1984) were able to show that even after prolonged tissue culture such ES cells have the capacity to colonize the germ line of chimeric mice when injected into blastocysts. Homologous recombination in mammalian cells between an artificial targeting vector and an endogenous gene was first achieved for the β-globin locus, although at a very low frequency, by Smithies et al. (Nature (1985) 317:230-234). These experiments made it possible to alter the mouse genome by homologous recombination in ES cells. Targeting of non-selectable genes became possible after enrichment strategies for homologous recombination were developed (e.g., use of selective markers). The murine immunoglobulin gene loci soon became targets for selective disruption. For example, Chen et al. (1993, International Immunology 5:647-56), generated mice that were not able to assemble immunoglobulin heavy chain genes. The procedures that successfully disrupted endogenous immunoglobulin gene loci paved the way for the transgenic mice described above that produce substantially human monoclonal antibodies through hybridoma technology.

Although the gene knockout procedure in mice is well known, immunoglobulin gene knockout techniques have not been perfected in birds. The equivalent method in birds is especially challenging because birds have an embryology and B cell diversity strategy very different from mice, humans and most mammals. First, unlike most mammals, avian species have only limited combinatorial diversity. For example, chickens have only single functional V and J gene segments at both the heavy and light chain locus. (Funk and Thompson (1996) Imm. and Dev. Bio. of the Chicken 17-28). To generate the varied repertoire necessary for an effective immune response, chicken B cells diversify their immunoglobulin genes during development in the bursa of Fabricius, an organ found only in bird species. The diversification strategy involves a process of somatic gene conversion, a DNA recombination process which involves unidirectional transfer of nucleotide sequence blocks. This gene conversion process for B cell diversity is only found in a small number of mammalian species. Therefore, because of fundamental biological differences between birds and mammals, and due to the absence of ES cell technology and genetic constructs for transgenesis, the potential for protein and antibody production in avian species has not been realized.

### SUMMARY OF THE INVENTION

The present invention is transgenic animals that produce non-endogenous proteins, specifically human immunoglobulins in response to antigen challenge, hybridomas derived from such animals, and compositions of monoclonal and polyclonal antibodies derived from the transgenic animal. The invention also includes methods of producing transgenic animals, specifically birds, and most specifically chickens, comprising a genetic modification that enable the expression of human antibodies and functionally disrupted endogenous immunoglobulin gene locus. The endogenous gene knockout may be created by transferring a targeting construct into a pluripotent cell such that the targeting construct combines with the native locus by homologous recombination and disrupts an endogenous locus thereby eliminating endogenous immunoglobulin molecule production. Alternate strategies for gene insertion and gene knockout specific to birds are described below.

Specifically included in the invention are specially designed targeting constructs to disrupt the endogenous immunoglobulin heavy chain locus in the chicken. Such targeting constructs include a transgene, preferably a positive-negative selection vector, which is constructed to target and delete a segment of the locus necessary for recombination. The functional disruption of a gene segment necessary for heavy chain rearrangement prevents the gene from rearranging to encode a heavy and a light immunoglobulin chain endogenous to the chicken. Endogenous gene segments necessary for rearrangement include variable (V), diversity (D), and joining (J) and constant (C) region gene segments. In another embodiment, the constant region is disrupted or deleted to prevent endogenous immunoglobulin production. In this aspect of the invention, the positive-negative selection vector is introduced into a cell line, such as an embryonic stem cell or chicken pre-B cell line, after which cells are selected where in the positive-negative selection vector has integrated into the genome of the chicken by way of homologous recombination. After transplantation of a pluripotent cell into a recipient embryo and breeding to homozygosity, the resultant transgenic chicken is substantially incapable of mounting an immunoglobulin—mediated immune response.

In another embodiment, the endogenous immunoglobulin heavy chain locus is completely deleted by site specific recombination at a point on the endogenous chromosome that is either located within the heavy chain locus itself, such as at a J region, or at a point centromeric to the entire heavy chain locus. Insertion of a specially designed genetic construct results in elimination of the endogenous locus, or a portion thereof, and replacement with a portion of the construct. The construct includes an entire exogenous chromosome containing an immunoglobulin locus that has been specifically modified to recombine with and become integral with the target avian chromosome. In a preferred embodiment, engineered genetic modifications are used to alter an endogenous avian chromosome 15. These modifications include the targeted disruption of the immunoglobulin light chain locus by homologous recombination, the targeted disruption of the immunoglobulin heavy chain locus by homologous recombination, the elimination of the endogenous heavy chain locus by site specific recombination and the replacement of the endogenous heavy chain locus through site specific recombination wherein a human immunoglobulin heavy chain locus derived from human chromosome 14 and/or a human light chain locus derived from human chromosome 22 or human chromosome 2 are added to avian chromosome 15 in cells having enhanced recombination proficiency, such as the avian pre-B cell line known as DT40 cells (see Fournier et al. U.S. Pat. No. 5,543,319). Through a transfer mechanism, such as microcell mediated chromosome transfer, the modified chromosome is transferred to a pluripotent cell such as an avian embryonic stem cell.

This invention also includes modified avian pluripotent cells, such as embryonic stem cells containing the modified chromosomes described herein, or native chromosomes comprised of the genetic modifications described herein. Use of transgenic techniques with long term avian ES cell cultures produce chimeric and transgenic birds derived from prolonged embryonic stem cell cultures. These cells are stable and exist in prolonged culture and are a source of pluripotent cells with genetic constructs that introduce a transgene comprising an unrearranged human immunoglobulin locus capable of rearrangement into the genome of a chicken enabling the production of human immunoglobulin molecules or fragments thereof. When combined with a host avian embryo by known procedures, those modified ES cells produce chimeric birds that incorporate the transgene into the somatic tissue of the resulting animals. These chimeric or transgenic birds exhibit an ES-cell derived phenotype and can express polyclonal human immunoglobulin molecules in response to antigen challenge. This invention includes compositions comprising long-term cultures of chicken embryonic stem cells that have been genetically modified to incorporate human immunoglobulin genes and which contribute to the somatic tissues and the germ line of recipient embryos. The modified embryonic stem cells include native endogenous chicken immunoglobulin loci having targeted disruptions of the immunoglobulin locus by homologous recombination, as well as native immunoglobulin loci that are deleted by homologous recombination to yield a modified avian chromosome that lacks an endogenous immunoglobulin locus, and which may be integral with one or more human immunoglobulin loci.

Modified avian chromosomes of the invention also include human immunoglobulin gene loci that are randomly integrated into the chicken genome in an unrearranged, germline configuration such that antigen challenge of a transgenic chicken having the human immunoglobulin loci in the somatic tissues or germline of the animal results in rearrangement of the human immunoglobulin genes and expression of polyclonal antibodies that are substantially human. In a particular embodiment, the methods and genetic constructs of the invention create an avian embryonic stem cell that is trisomic for chicken chromosome 15 wherein two of the three copies of avian chromosome 15 are endogenous and one is a modified chromosome comprised of human immunoglobulin loci. Thus, the present invention includes sustained embryonic stem cell culture having targeted disruptions or deletions of the endogenous immunoglobulin locus, and/or having an immunoglobulin locus of human origin.

Because an important advantage of the avian model is the concentration of polyclonal immunoglobulin molecules in the egg, the invention also includes the eggs produced by the transgenic chickens that result from the methods of this invention and which contain human immunoglobulins, including specifically human polyclonal immunoglobulins that specifically bind to an antigen with which the transgenic animal of the invention is immunized.

### DESCRIPTION OF THE FIGURES

FIG. 1 is a diagram of the pCX/GFP/Puro plasmid construct used for transfection of ES cells.

FIG. 2 is a FACS analysis of non-transfected chicken ES cells (upper panel) and chicken ES cells that have been transfected with the pCX/GFP/Puro construct and grown in the presence of puromycin. The analysis shows that substantially all of the transfected cells are expressing the transgene.

FIG. 3 is a Southern analysis of ES cells that have been transfected with the pCX/GFP/Puro construct. The difference in the location of the probe in preparations of DNA digested with BamH1, EcoR1 and a combination of the two endonucleases indicates that the transgene is incorporated into the genome at different sites in the cell lines TB01 and TB09.

FIG. 4 is a schematic of pKO scrambler targeting vector used for the functional disruption or knockout of the endogenous avian immunoglobulin genes.

FIG. 5 is a schematic of a construct for the functional disruption or knockout of the endogenous avian immunoglobulin heavy chain gene.

FIG. 6 is a schematic of a construct for the functional disruption or knockout of the endogenous avian immunoglobulin light chain gene.

FIG. 7 is a schematic of an alternate construct for the functional disruption or knockout of the endogenous avian immunoglobulin light chain gene.

FIG. 8 is a schematic of an embodiment of a method of the invention for producing a modified avian chromosome encoding human immunoglobulins from an unrearranged immunoglobulin locus, wherein the chromosome is assembled in a DT40 cell and introduced into a chicken embryonic stem cell.

FIG. 9 is the PCR analysis for human VJ rearrangements of Igκ BAC in chicken B cells.

FIG. 10 shows the construction of simplified human IgMk minilocus constructs. The source of each part of the construct, human or chicken, is designated under the line. The location of the chicken 3′ enhancer is shown.

### DETAILED DESCRIPTION OF THE INVENTION

The two major components of the vertebrate immune system are B and T lymphocytes. The B cells are responsible for producing very specific proteins called antibodies, or immunoglobulins, that protect the organism from antigens. Immunoglobulins are large molecules composed of two identical light (L) polypeptide chains and two identical heavy (H) chains, held together by disulfide bonds. Each polypeptide chain has a variable (V) and a constant (C) region of amino acid sequences. The variable regions recognize the antigens and thereby give specificity to the immunoglobulin.

To produce an effective immune response, the immune system must produce a large number of distinct immunoglobulin molecules. However, most B cells only produce a single antibody specificity. Thus, an effective immune repertoire requires a vast repertoire of B cells, only a few of which will ever encounter their cognate antigen. Some mammals that have been extensively studied use a characteristic method to create a diverse immunoglobulin repertoire. These mammals have an immunoglobulin gene locus which contains multiple functional (V), diversity (D) and joining (J) gene segments that can be recombined during B cell development to generate an immunoglobulin gene. By combining distinct assemblies of V(D)J antigen binding exons, the B cell repertoire creates a varied set of immunoglobulin molecules. In addition, the independent rearrangement and association of immunoglobulin heavy and light chains within a single cell further increases the spectrum of antibody specificities.

The human polyclonal antibodies of the present invention bind to virtually any substance that is antigenic to the transgenic bird. To produce high affinity isotype G antibodies, the transgenic chicken must undergo an immune response that produces the antibodies and permits transport of the antibodies into the egg yolk of the animal. To achieve this, the human immunoglobulin genes inserted into the genome of the bird must undergo rearrangement to produce the complete repertoire of antibody molecules and must permit diversification of the B cell repertoire and interaction with the other components of the endogenous immune system to express high affinity antibodies from a mature B cell repertoire. As a result, the construction of the human immunoglobulin loci in the transgenic bird is achieved by selecting appropriate portions of the DNA encoding the immunoglobulin molecules as well as the DNA allowing for the gene rearrangement processes.

In the transgenic bird, the heavy and light chains of the immunoglobulin molecule are encoded by human immunoglobulin loci that are specially designed to be expressed in the B lymphocyte cell lineage. Specifically, the human constant region is preserved throughout the cμ to C-gamma coding regions to maximize recombinational potential and to provide a human constant region locus that is equivalent in size to the native locus in humans. Furthermore, in the embodiment described herein, wherein modifications to chromosome 15 are performed to place the human heavy chain and light chain loci in a tightly linked configuration on an avian chromosome, the configuration is unique in that both loci are present on the same chromosome, which is not the native state in humans. The placement of the two immunoglobulin loci on the same chromosome offers discrete advantages in breeding animals to homozygosity. If the immunoglobulin loci are located on separate chromosomes, the breeding of double heterozygotes has a one-in-sixteen chance of producing an animal that is homozygous for both heavy and light immunoglobulin genes. However, when the heavy and light loci are present on the same avian chromosome, the tightly linked configuration increases the chances of homozygosity to almost one-in-four.

In humans, immunoglobulin heavy chains are encoded by the immunoglobulin heavy chain locus located on human chromosome 14. The two classes of immunoglobulin light chains, kappa and lambda, are located on human chromosomes 22 and 2, respectively. The human heavy chain locus is comprised of a large number (approximately 100 or more) V region gene segments, 12 D segments, 4 J segments, and 8 C segments. The J cluster genes cover approximately 1 kb of DNA and are separated by the C mu gene by an intron approximately 6 kb. The C mu gene is the constant region segment most proximate to the J region. Transcriptional enhancers and switch regions are located between the J segments and the C segments and switch regions are also located immediately 5′ of each of the constant coding regions for each heavy chain isotype (such as immunoglobulin-G, IgE, IgD, etc.). The kappa locus consists of a large number of V regions and approximately 5 J regions with a single constant segment. The large number of V segments can be grouped into a smaller number of families whose function and orientation is not yet determined. The lambda locus is comprised of a constant segment, with a dedicated J region and wherein the distance between the paired V and C segments are inversely proportional to the observed rearranged frequency.

In aves, immunoglobulin gene rearrangement occurs throughout development and differentiation as described above in connection with the bursa of Fabricius. In addition to the anatomical differences in the sites of B cell development in birds and mammals, there are also differences in the apparent regulation of immunoglobulin rearrangement. In mammals, immunoglobulin heavy and light chain gene rearrangements occur in an ordered fashion wherein heavy chain gene loci rearrange first with the initial step being the joining of a D segment to a J segment. After D-J heavy chain rearrangements have occurred, heavy chain variable gene segments recombine with diversity segments to yield a V-D-J rearrangement. The V-D-J subunit is joined with a constant region, initially a mu constant region, and the IgM heavy chain is expressed. Only after a proper series of immunoglobulin gene rearrangement yields an immunoglobulin molecule with a competent mu chain, can the light chain be expressed. In birds, however, the light and heavy chain genes rearrange simultaneously and B cells can be found in which the light chain has rearranged in the absence of complete light chain rearrangement.

In both birds and mammals, productive light and heavy chain locus rearrangements allow the B cells to assemble a complete immunoglobulin class M (IgM) molecule which is then either secreted or expressed on the membrane of the B cell. A population of B cells expressing IgM immunoglobulins on the cell surface is referred to as a primary repertoire. The membrane bound form of the IgM molecule serves as a receptor to facilitate clonal expansion in response to antigen stimulation. The B cell progeny that have carried out productive rearrangements of both heavy and light chain genes continue to produce antibody of the specificity determined by the V regions. Certain B cells will produce antibody of different isotypes and different immunoglobulin heavy chain classes, while retaining the same variable regions. This process yields the secondary B cell repertoire and exhibits both class switching and somatic mutation or gene conversion to yield higher affinity antibodies.

In both birds and mammals, immunoglobulin heavy chain genes undergo a distinct rearrangement separate from the V-D-J recombination to express different classes or isotypes of immunoglobulin heavy chain. Class switching results in the shifting of a rearranged V-D-J complex from a mu isotype heavy chain to a non-mu downstream constant region. In mammals, there are at least five isotypes of the immunoglobulin molecule, namely IgM, IgG1-4, IgA, IgD and IgE whereas in chickens only 3 immunoglobulin isotypes, namely IgM, IgY and IgA have been identified. Each constant region isotype has a switch region immediately 5′ thereof to facilitate class switch recombination. In vertebrates, switch regions consist of randomly repeated short sequences of DNA that are shared by all switch regions as well as other repeated units that are more or less unique to the specific switch region for the constant region isotype. The specific switch sequences found in humans, mouse, and chickens are slightly different, but all are believed to be derived from common primordial switch repeats as described by Kitao et al. (2000, International Immunology 12, 959-968).

As described in more detail below, the region between the C mu constant region and the gamma switch region is a unique feature of the transgenic bird of the present invention. In earlier transgenic animals, the region between the C mu coding region and the gamma switch region was intentionally removed when constructing the human immunoglobulin heavy chain transgene. Despite the removal of this DNA, to yield an engineered immunoglobulin heavy chain locus that is shorter than the native locus, the two respective isotypes (mu and gamma) were not co-expressed, but were separately and exclusively expressed by discrete B cell repertoires with evidence of somatic mutation accompanying class switching and occurring as point mutations in the variable region relative to the unrearranged locus. Class switching and somatic mutation have not been demonstrated in a transgenic animal having an exogenous human heavy chain locus with a C mu-C gamma locus of an equivalent size or larger, as compared to the native human locus.

As described in the following examples, the immunoglobulin locus of the present invention that is integrated into the avian germline is comprised of an immunoglobulin heavy chain gene locus that is as large as, or larger than, the native locus and, specifically with respect to the region between the mu coding and the gamma coding region, the size of the locus is as large as or greater than the locus in a native unrearranged human immunoglobulin gene. Pursuant to this invention, the region between the mu constant region and the gamma constant region is preserved to enhance the recombination capabilities of the exogenous human immunoglobulin locus in the transgenic bird.

The bursa is a unique organ, found only in birds, which arises at day 5 of embryonic life. (Weill and Reynaud (1987) Science 238:1094-98). Its removal during early embryonic development (up to day 17 of incubation) prevents the animal from mounting an immune response to any immunizing antigen. Bursal development involves two phases. The first is the intraembryonic phase which includes the colonization and the growth of about 10⁴ bursal follicles by expansion of their B cell clones. The second phase is the posthatching period which includes the seeding of bursal cells to the periphery and the continuous expansion of the bursal follicles. By the time the hatched chick is four weeks old, a sufficient number of stem cells has migrated out of the bursa as postbursal stem cells, thus installing the mature chicken B cell immune system in the periphery.

In a transgenic animal of the invention exhibiting a complete absence of functional endogenous immunoglobulin gene rearrangement, the bursa will not develop normally, but will have characteristic abnormalities indicative of the gene disruption. Thus, a homozygous JH region knockout would have a bursa that was not populated with B cells and has no clear follicular structure and is physically much smaller than a normal bursa. The engineered gene disruption dissociates any possible transcriptional/translational start from the constant region coding sequence and results in a complete lack of immunoglobulin production in the homozygous knockout animal.

However, where the bird is producing human immunoglobulins from a functionally rearranged immunoglobulin locus, the bursa will be populated with avian B cells expressing human immunoglobulins and would have a follicular structure that is phenotypically normal.

The chicken immunoglobulin-L locus encodes a single functional V_{L} gene segment separated by 1.8 kb from a single functional J_{L} gene segment. A single C_{L} region is located 2 kb 3′ from the J_{L} segment. The functional V_{L} segment, designated V_{L1}, is split in the leader region by a 125-bp intron, and the promoter region of V_{L1} includes a conserved octomer box 32 bp upstream from the TATA box. In a 22-kb region upstream of V_{L1}, there are 25 V_{L}-homologous gene segments situated in both transcriptional orientations. All 25 of these V_{L} gene segments are truncated at the 5′ end and lack a leader exon and promoter region. In addition, all lack a functional recombination signal sequence (heptamer-spacer-nonamer) at the 3′ end and are not capable of V-J rearrangement. These 25 gene segments are designated as V_{L} pseudogenes, ψV_{L} I-25.

The chicken immunoglobulin-H locus is also restricted in its capacity for combinatorial diversity. The immunoglobulin-H locus consists of a single functional V_{H1} segment located 15 kb 5′ from a single functional J_{H} gene segment, with sixteen D_{H} segments between V_{H1} and J_{H}. There is little sequence variation between germline D_{H} segments, thereby limiting combinatorial diversity. A cluster of 80-100 V_{H} pseudogene segments (ψV_{H}), spanning a region of 60-80 kb, is present 5′ of the functional V_{H1} gene. As in the case of the ψV_{L} segments, the ψV_{H} segments lack a promoter region, leader exon, and recombination signal sequences. Many of the ψV_{H} segments are situated with alternating transcriptional orientation.

The single V and J segments are rearranged by V(D)J recombination during a brief period of early chicken B cell development creating only limited diversity at the junction of the V and J gene segment. Further diversity of the rearranged V gene is acquired during B cell proliferation in the bursa. There, blocks of pseudogene sequences appear in the rearranged V gene, whereas the sequences of the pseudogenes and the unrearranged V gene segment do not change. This nonreciprocal transfer of sequence information from the pseudogenes into the rearranged V gene was named gene conversion in analogy to similar processes in yeast.

The conversion tracts comprise from 10 to more than 120 bp, and a single V gene can receive segmental exchanged from up to six different pseudogenes. The number of events increases with the time that the B cells spend in the bursal environment, consistent with the idea that gene conversion occurs in a stochastic manner, with more events accumulating as the number of cell divisions increases. It has been estimated that one successful conversion event occurs every 10 to 15 cell divisions.

The frequency of usage of the ψV segments for conversion events appears to depend on a number of variables. First, pseudogene segments proximal to the V gene are used more frequently than distal ones. Second, ψV segments in the antisense orientation are used preferentially over segments in the sense orientation. Finally, sequence homology seems to be important for the reaction, since pseudogenes with greater sequence similarity to the V gene serve more often as donors.

As noted above, to produce an animal whose immunoglobulin production is exclusively human, the endogenous immunoglobulin production is eliminated. The techniques for disruption of endogenous immunoglobulin expression include transferring a specially designed construct into a cell, such that the construct becomes integrated into the germline of the animal and ultimately results in the disruption of the production of endogenous immunoglobulin molecules. The disruption of endogenous immunoglobulin production may occur by targeted disruption of a specific immunoglobulin gene locus, the substantial removal of an entire endogenous immunoglobulin gene locus, or the removal of a locus in combination with the insertion of an engineered construct containing an exogenous locus. These steps may be performed in an embryonic stem cell or in another cell type that facilitates genetic recombination prior to transfer into a pluripotent cell.

In one embodiment of the invention, the species is a bird having disrupted immunoglobulin production such that, when challenged with antigen, essentially no endogenous antibody production results. In another embodiment, the bird may express non-avian immunoglobulin molecules encoded by specifically engineered non-avian constructs incorporated into the germline DNA of the ave. These constructs may or may not also cause the disruption of endogenous immunoglobulin production.

In one embodiment, the present invention is a transgenic chicken produced by introducing a targeting construct comprising at least one selectable marker and at least one homologous portion of the chicken immunoglobulin gene into a recombination proficient cell line, such as the avian pre-B cell line designated DT40, disrupting the endogenous immunoglobulin gene in the DT40 cell by homologous recombination, making microcells from the disrupted DT40 cell, fusing the microcells with chicken embryonic stem (cES) cells, selecting cES cells carrying the targeted immunoglobulin locus and creating a chimeric chicken that contains the disrupted immunoglobulin locus. The disrupted immunoglobulin locus is inherited by donor-derived offspring of the chimeras and is bred to homozygosity using techniques known in the art. Birds that are homozygous for the disrupted immunoglobulin locus produce negligible amounts of the endogenous immunoglobulin.

In another embodiment, a targeting region comprising at least two regions of homology and a selectable marker is introduced directly into an embryonic stem cell wherein the construct deletes a portion of the endogenous locus through homologous recombination. Preferably, the targeting vector is a positive-negative selection type vector to facilitate selection of successfully transformed embryonic stem cells. This approach may be applied to the functional disruption of both the endogenous heavy and light chain immunogloblin genes directly in embryonic stem cells, as well as in DT40 or other recombination proficient cell lines.

Also included in the invention are specially designed constructs to disrupt the production of endogenous immunoglobulin production in the chicken and, in specific embodiments, the disruption of an endogenous locus or the insertion of a construct comprising a defective locus that is incapable of functional rearrangement of the immunoglobulin genes. Such targeting constructs and methods of their production utilize a transgene comprising a gene targeting vector, preferably a positive-negative selection vector, that targets the endogenous locus by homologous recombination yielding the functional disruption of a selected gene or a class of gene segments encoding a heavy and/or light endogenous immunoglobulin chain gene. Endogenous gene segments suitable for targeting include the diversity, joining and constant region gene segments necessary for gene rearrangement, as well as combinations of these. For functional disruption of the endogenous heavy chain, the deletion of the J segments are preferred to eliminate the possibility for a productive V-D-J rearrangement in coding an endogenous heavy chain variable region. However, constant region disruption through deletion or insertion is also possible as is deletion of multiple gene segments, especially in the light chain gene. As described below, immunoglobulin loci that are proximate to a telomere of a chromosome may be deleted in their entirety through genetic recombination with several types of specially engineered constructs.

The targeting vector construct is comprised of regions of homology to the endogenous immunoglobulin locus and one or more markers that identify embryonic stem cells that have been successfully targeted by the vector. After recombination, the endogenous locus is rendered non-functional by the deletion of elements required for recombination, such as a V, D, J, or C region, or may have the insertion of one or more sequences, such as a stop codon that prevents expression of a partially or totally rearranged locus. In this aspect, the invention includes embryonic stem cells comprised of gene loci targeted with a positive-negative selection vector wherein the vector has integrated into the genome of the chicken by way of homologous recombination and the endogenous locus is characterized by the presence of elements of the targeting vector. After transplantation of these pluripotent cells into recipient embryos and breeding to homozygosity by techniques known in the art, the resultant transgenic chicken is substantially incapable of endogenous immunoglobulin gene rearrangement and gene expression, and is incapable of mounting an immunoglobulin mediated immune response as a result of the integration of the vector into the avian chromosomal DNA.

In another embodiment, the endogenous immunoglobulin heavy chain gene is located at a site that is proximate to the telomere of an identified avian chromosome. The location of the heavy chain locus at the telomeric end of a chromosome provides the ability to target the locus through either homologous or site specific recombination. The ability to target this location for the immunoglobulin heavy chain knockout, is a function of the proximity of the locus to the telomere of the chromosome and the necessity of the region of DNA that is telomeric to the locus. Depending on the organism, if the telomeric DNA is not necessary for the survival of the organism, such that the deletion of all DNA telomeric of the immunoglobulin heavy chain locus results in a non-lethal mutation, then the disruption of the immunoglobulin heavy chain may be achieved by a recombination event that is centromeric to the immunoglobulin heavy chain locus, or which occurs within the locus such that gene rearrangement is effectively precluded. In this embodiment, the methodology includes insertion of a recombination site centromeric to the immunoglobulin heavy chain locus or, as noted above, within the locus such that recombination is prevented.

Once the endogenous locus has been so modified, a construct can be introduced to the endogenous chromosome to cause site-specific recombination. In this embodiment, the construct of the invention includes a complementary recombination site to yield a recombination event centromeric to or within the region of DNA comprised of the immunoglobulin heavy chain gene. In a preferred version of this embodiment, the chromosome is avian chromosome 15 and site specific recombination is achieved at a engineered recombination site centromeric to the entire chicken immunoglobulin heavy chain locus, or at a site necessary for recombination such as the J segments, and the construct contains a complimentary recombination site attached to a segment of DNA comprised of at least one human immunoglobulin locus. In a preferred embodiment, the human immunoglobulin light chain lamda locus and the human immunoglobulin heavy chain locus are each constructed to contain a first and a second site-specific recombination.

In this embodiment, a recombination site is first inserted into chicken chromosome 15 centromeric to the chicken immunoglobulin heavy chain locus. A construct, which may comprise an entire human chromosome containing a recombination site and the human immunoglobulin locus, is introduced to the avian chromosome under conditions causing recombination between the two recombination sites and the construct replaces all of the endogenous DNA that is telomeric to the recombination site. In this fashion, the chicken immunoglobulin heavy chain gene can be deleted and replaced with a construct of choice. The construct, either before or after integration into the avian chromosome, may also contain a second recombination site that is telomeric to the unrearranged human immunoglobulin light chain locus. In this configuration, the modified avian chromosome 15 contains a first recombination site centromeric to the human immunoglobulin light chain lambda locus and a second dissimilar recombination site telomeric of the lambda locus.

This configuration is suited for reaction with a second construct which may comprise human chromosome 14 comprising the human immunoglobulin heavy chain locus and the second recombination site. Placed under conditions suitable for recombination of the second recombination sites, the human immunoglobulin heavy chain locus is integrated into avian chromosome 15 at a site telomeric of the human immunoglobulin light chain lamda locus. In a similar fashion, the human immunoglobulin light chain kappa locus may be integrated into avian chromosome 15 in an orientation compatible with the lamda locus. The heavy chain knockout of this embodiment is preferably achieved in a recombination-proficient cell line such as an avian pre-B cell DT40 cell line or equivalent. Where the functional disruption is achieved by direct targeting of the endogenous locus with a homologous recombination type targeting vector, the method may be performed in such recombination-proficient cells lines or directly in pluripotent cells exhibiting an ES cell phenotype.

Stable, long-term cultures of ES cells are necessary to produce chickens that deposit rearranged human antibodies in their eggs, and to make site-specific changes to the avian genome to disrupt endogenous immunoglobulin production. The development of chimeric or transgenic avians requires that chicken embryonic stem (cES) cell lines be created that contribute to somatic tissues when injected into a recipient embryo. Specifically, the embryonic stem cell cultures are sustained for an extended length of time during which desirable phenotypes in chimeric animals resulting from the injection of embryonic stem cells can be identified, and during which genetic modifications can be made to the genome of the embryonic stem cell to introduce human immunoglobulin genes into a chimeric offspring. In preferred embodiments, avian embryonic stem cell cultures are maintained for an extended period of time and can be engineered to contain transgenes comprising unrearranged genes encoding human immunoglobulin repertoire including isotype gamma (IgG) antibodies.

Chicken ES cell lines are derived from stage X embryos and have a large nucleus and contain a prominent nucleolus. These cells are confirmed to be chicken embryonic stem (cES) cells by morphology after long-term culturing and to yield chimeras when injected into recipient embryos. Moreover, the ES cells enable a high degree of contribution to somatic tissues as determined by extensive feather chimerism. Still further, these embryonic stem cells are demonstrated to be transfected with transgenes carrying DNA encoding and exogenous protein. The ES cells stably integrate the transgene and express the transgene to enable selection of transformed cells. These transformed cells are capable of forming chimeras wherein an exogenous protein encoded by the transgene is present in the germline and somatic tissue of the chimera, and that cells derived from the chimera express the exogenous protein encoded by the transgene. Embryonic stem cell progeny are derivatives of ES cells that differentiate into non-ES cell phenotypes after introduction of the ES cells into recipient embryos and the formation of a chimera. A transgenic chicken is the progeny of a chimera which has been produced from chicken ES cells carrying a transgene which is stably integrated into the genome when cells derived from the transgenic ES cells have incorporated into the germline.

Expression of the transgene in somatic tissue is demonstrated in extraembryonic and somatic tissues including the allantois, endoderm, mesoderm, and ectoderm of the transgenic animal and is broadly expressed in all tissues and organ types. In a preferred embodiment, the CX promoter is used to facilitate the broad expression of green fluorescent protein (GFP) in somatic and germline tissues of a transgenic chicken. Analysis of the haemopoetic lineage of transgenic animals demonstrates expression in the B lymphocytes of the immune system cells of an exogenous protein encoded by the transgene described herein using the long-term embryonic stem cells of the present invention. In the preferred embodiment, human immunoglobulin molecules, or fragments thereof, are encoded by the transgene and the rearranged transgene is detected in cells of the B-lymphocytes lineage. In a preferred embodiment, the constituents of the human immunoglobulin locus are operably linked to B lymphocyte specific regulator regions, including promoters, enhancers, CIS acting elements, transacting elements, and silencers.

Chicken ES cells were derived from one of two crosses: Barred Rock X Barred Rock or Barred Rock X Rhode Island Red. These breeds were selected to obtain a feather marker when testing the developmental potential of cES cells. The cES cells are injected into White Leghorn embryos, which are homozygous dominant at the dominant, white locus. Chimeric chickens resulting from injection of these ES cells display black feathers from the cES cells and white feathers from the recipient embryo.

Initial establishment of the cES cell culture was initiated according to the protocol developed by J. Petitte, see U.S. Pat. No. 5,565,479, which is specifically incorporated herein by reference. At stage X, the embryo is a small round disk, consisting of approximately 40,000-60,000 cells, situated on the surface of the yolk. To retrieve the embryo, a paper ring is put on the yolk membrane, exposing the embryo in the middle. The yolk membrane is cut around the ring, which is then lifted off the yolk. The embryo, attached to the ventral side of the ring, is placed under the microscope and the area pellucida isolated from the area opaca using a fine loop.

TABLE 1
cES cell lines derived on either STO feeder cells or a polyester
insert in CES-80 medium. The cultures were initiated
from both single and pooled embryos.

| Cell | Donor | Substrate used to | |
|---|---|---|---|
| line | embryo | derive cES cells | Endpoint of cell line |
| 009 | pooled | STO | cultured for 3 months, injected & |
| | | | cryopreserved |
| 029 | pooled | insert | cultured for over 3 months, injected & |
| | | | cryopreserved |
| 31 | pooled | STO | injected at 4 days |
| 36 | pooled | STO | injected at 13 days |
| 50 | pooled | STO | grown for over 8 months, injected & |
| | | | cryopreserved |
| 63b | pooled | insert | grown for 3 months and cryopreserved |
| 671 | single | insert | injected at 45 days of culture |
| 307 | pooled | STO | injected at 15 days and fixed for staining |
| 314 | pooled | STO | cultured for over 3 months, injected & |
| | | | cryopreserved |
| 317 | pooled | STO | injected at 12 days and fixed for staining |
| 324A | single | insert | cultured for over 6 months and injected |
| 328 | single | insert | cultured for over 6 months, injected & |
| | | | cryopreserved |
| 329 | single | insert | cultured for 5 months, injected & |
| | | | cryopreserved |
| 330 | single | insert | cultured for 3 months and cryopreserved |
| 331 | single | 24 w insert | cultured for over 3 months and terminated |
| 332 | single | 96 w STO | cultured for 3 months and cryopreserved |
| 333 | single | 12 w insert | cultured for over 3 months and terminated |
| 334 | single | 12 w insert | cultured for over 3 months and terminated |
| 335 | single | 96 w insert | cultured for over 3 months and terminated |

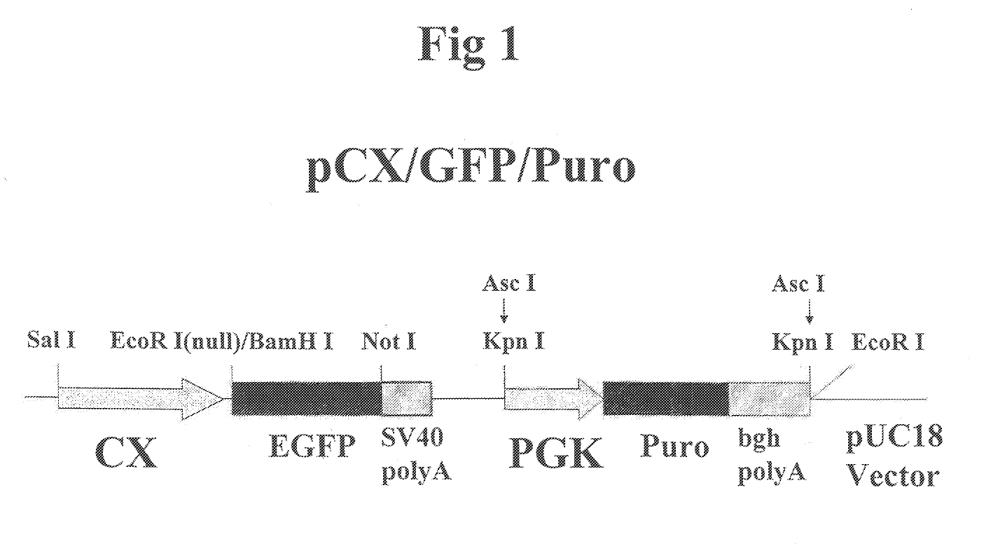

Embryos are dispersed mechanically into a single cell suspension and seeded on a confluent feeder layer of mitotically inactivated STO cells at a concentration of 3×10⁴ cells/cm². The cES culture medium consists of DMEM (20% fresh medium and 80% conditioned medium) supplemented with 10% FCS, 1% pen/strep; 2 mM glutamine, 1 mM pyruvate, 1× nucleosides, 1× non-essential amino acids and 0.1 mM β-mercaptoethanol. Before use, the DMEM medium is conditioned on Buffalo Rat Liver (BRL) cells. Briefly, after BRL cells are grown to confluency, DMEM containing 5% serum is added and conditioned for three days. The medium is removed and a new batch of medium conditioned for three days and repeated. The three batches are combined and used to make the cES medium. Chicken ES cells become visible 3-7 days after seeding of the blastodermal cells. These cES cells were morphologically similar to mES cells; the cells were small with a large nucleus and a pronounced nucleolus (See FIG. 1).

The growth characteristics of cES cells are different from mES cells, which grow in tight round colonies with smooth edges and individual cells that are difficult to distinguish. Chicken ES cells grow in single layer colonies with clearly visible individual cells. Tight colonies are often the first sign of differentiation in a cES culture.

To test for markers of pluripotency of the cells that were derived in culture, the cells were fixed and stained with SSEA-1 (Solter, D. and B. B. Knowles, Proc. Natl. Acad. Sci, U.S.A. 75: 5565-5569, 1978), and EMA-1, which recognize epitopes on primordial germ cells in mice and chickens (Hahnel, A. C. and E. M. Eddy, Gamete Research 15: 25-34, 1986), and alkaline phosphatase (AP) which is also expressed by pluripotential cells. The results of these tests demonstrate that chicken ES cells express alkaline phosphatase and the antigens recognized by SSEA-1 and EMA-1.

Although cES cells are visible after using the above protocol, such cultures cannot be maintained longer than a few weeks. Several modifications in culture conditions were initiated, as discussed below, which led to the derivation of 19 cell lines (Table 1) of which 14 were tested for their developmental potential by injection into recipient embryos. Eleven of the 14 cell lines contributed to recipient embryos as determined by feather pigmentation (See Table 2 below). This protocol yields sustained cultures of pluripotent cells expressing an embryonic stem cell phenotype. At any point, the cells can be cryopreserved and when injected into compromised recipient embryos have the potential to substantially contribute to somatic tissues.

TABLE 2
Passage number and time in culture of embryonic stem cell lines derived from single
or pooled embryos. Frequency and extent of somatic chimerism after injection of these
cES cells into stage X recipients.

| | | | time in | # of | | | | Extent of |
|---|---|---|---|---|---|---|---|---|
| Cell | Donor | Passage | culture | embryos | # | # | % | chimerism¹ |
| line | embryo | number | (days) | injected | chimeras | analyzed | chimeras | (%) |
| 31 | pooled | 0 | 4 | 15 | 2 | 7 | 28.5 | 1-5 |
| 317 | pooled | 4 | 12 | 29 | 2 | 10 | 20 | 25-65 |
| 36 | Pooled | 1 | 13 | 24 | 1 | 5 | 20 | 15 |
| 307 | pooled | 4 | 15 | 21 | 1 | 6 | 17 | 5 |
| 330 | single | 6 | 33 | 11 | 3 | 8 | 25 | 5-50 |
| 63b | pooled | 11 | 72 | 36 | 4 | 21 | 19 | 1-10 |
| 671 | single | 3 | 45 | 28 | 0 | 15 | 0 | — |
| 324A | single | 10 | 65 | 25 | 0 | 15 | 0 | — |
| 009 | pooled | 20 | 61 | 27 | 0 | 9 | 0 | — |
| 329 | single | 3 | 15 | 31 | 8 | 17 | 47 | 3-75 |
| 329 | | 6 | 25 | 30 | 9 | 19 | 47 | 3-95 |
| 329 | | 6 | 28 | 26 | 1 | 12 | 8 | 23 |
| 329 | | 11 | 49 | 10 | 1 | 4 | 25 | 60 |
| 029 | pooled | 4 | 33 | 40 | 9 | 27 | 33 | 5-80 |
| 029 | | 9 | 37 | 40 | 4 | 15 | 27 | 4-15 |
| 328 | Single | 6 | 56 | 19 | 4 | 11 | 36 | 10-80 |
| 328 | | 12 | 98 | 33 | 7 | 22 | 32 | 5-50 |
| 314 | Pooled | 17 | 52 | 30 | 2 | 5 | 40 | 5-65 |
| 314 | | 15-17 | 53 | 29 | 1 | 4 | 25 | 30 |
| 314 | | 17 | 55 | 37 | 3 | 15 | 30 | 3-80 |
| 314 | | 16 | 65 | 27 | 2 | 11 | 18 | 5-40 |
| 314 | | 14 | 61 | 25 | 0 | 13 | 0 | — |
| 314 | | 16 | 65 | 32 | 3 | 14 | 21 | 10-60 |
| 314 | | 20 | 61 | 30 | 4 | 5 | 80 | 4-50 |
| 314 | | 21 | 67 | 30 | 2 | 11 | 18 | 5-15 |
| 314 | | 21 | 71 | 8 | 0 | 2 | 0 | — |
| 50 | pooled | 7 | 53 | 35 | 7 | 23 | 30 | 4-65 |
| 50 | | 14 | 106 | 36 | 3 | 21 | 14 | 10-30 |

¹Extent of chimerism was determined by the proportion of black feathers.

As with the mouse, avian embryonic stem cells, which are sometimes referred to as embryonic germ cells, are derived on a variety of feeder layers including STO, STO-sn1 and others that are readily available. Leukemia Inhibitory Factor (LIF) produced by these feeders, and the addition of fetal bovine serum contributes to the maintenance of ES cells in an undifferentiated state. In a preferred embodiment of this invention, chicken ES cell cultures are initiated on a STO feeder layer. STO cells are grown to confluency, treated with 10 μg/ml mitomycin for 3-4 hours, washed, trypsinized and seeded on gelatin coated dishes at 4×10⁴ cells/cm². cES cells appear to grow more rapidly when the feeder of STO cells are sparser. Reducing the STO feeder cell concentration to between 10³ and 10⁵, and preferably below 10⁴ cells/cm², facilitates the derivation and propagation of cES cells. However, when chicken embryonic fibroblasts and mouse primary fibroblasts are used as feeders, no cES cells were derived. Also, when previously established cES cells were plated on these feeders, all of them differentiated within 1 week.

Growing cES cells on synthetic inserts, such as polymer membranes (Costar, Transwell type) in the absence of feeders avoids contamination of the recipient embryo with feeder cells when the ES cells are injected. As Table 3 and 4 show, culturing on inserts, instead of STO feeders, facilitates the derivation of cES cells, and inserts can be used for initial derivation. However, after initially growing rapidly on inserts, the mitotic activity of the ES cells declines after 4-6 weeks of culture. To extend the culture the cells have to be transferred to a feeder of STO cells.

TABLE 3
Establishment of cES cells from single embryos
on either inserts or a feeder of STO
cells (10⁴ cells/cm²).

| | # of cultures | # of cell lines |
|---|---|---|
| Substrate | started | obtained |
| STO feeder | 56 | 3 (5%) |
| Insert | 45 | 7 (16%) |

TABLE 4
Establishment of cES cells from pooled embryos
on either a STO feeder or a synthetic
insert.

| | # of cultures | # of cell lines |
|---|---|---|
| Substrate | started | obtained |
| STO feeder | 73 | 7 (9.5%) |
| insert | 17 | 2 (12%) |

The data in Tables 3 and 4 show that chicken embryonic feeder cells and mouse primary fetal fibroblasts do not support the derivation or the culture of cES cells. A feeder of STO cells supports derivation and growth but only when present in a limited concentration of between 10³ and 10⁵ STO cells but preferably in the present embodiment at a concentration of less than or appropriately 10⁴ cells/cm². A dense STO feeder layer impairs the growth of cES cells, while the specified concentration of STO cells provides factor(s) necessary for ES cell proliferation. When the cells are sustained over an extended culture period and continue to express an embryonic stem cell phenotype, and differentiate into non-embryonic stem cell phenotypes in vivo, they are referred to as “ES cell progeny.”

The cES cell culture medium consists of 80% conditioned medium and preferably contains certain BRL conditioned medium with factors necessary for the derivation and growth of cES cells. At a concentration of 50% conditional medium, growth of the cES cells is not as reliable as in 80% conditioned medium. When the percentage of conditioned medium is reduced to less than 50%, the growth of the cES cells is affected, as evidenced by an increase in differentiated cells and, at a concentration of 30% or less, the cES cells differentiate within 1 week. Also, the conditioned medium found necessary for the derivation and maintenance of cES cells does not maintain mouse ES but causes their differentiation.

Fetal bovine serum is a preferred component of the ES cell medium according to the present invention and contains factors that keep cES cells in an undifferentiated state. However, serum is also known to contain factors that induce differentiation. Commercially available serum lots are routinely tested by users for their potential to keep ES cells in an undifferentiated state. Serum used for cES cell cultures are known to differ from serum used for mouse ES cell cultures. For example, serum used for the culture of mouse ES cells that is low in cytotoxin and hemoglobin concentration, which is known to maintain mouse ES cells in an undifferentiated state, did not support the sustained growth of chicken ES cells.

Therefore, serum to be used on chicken ES cells should not be tested on mouse ES cells to determine suitability as a media component, but instead should be evaluated on chicken ES cells. To do so, chicken ES cell cultures are divided into two aliquots and used to test each new batch of serum. The new batch tested must clearly support the growth of chicken ES cells when empirically tested.

Chicken chromosomal spreads require special evaluation techniques different than mice because the complex karyotype consists of 10 macrochromosomes, 66 micro-chromosomes and a pair of sex chromosomes (ZZ in males and ZW in females). Long-term cES cells analyzed after 189 days in culture, and after being cyopreserved twice, exhibit a normal karyotype with 10 macro chromosomes; 2 Z-chromosomes and 66 microchromosomes.

Chicken ES cells are cryopreserved in 10% DMSO in medium. After thawing and injecting several cell lines into recipient embryos, somatic chimeras are obtained, indicating that the cES cells maintain their developmental potential during the cryopreservation process.

To permit access to the embryo in a freshly laid egg the shell must be breeched, inevitably leading to a reduction in the hatch rate at the end of the 21-day incubation period. The convention was to cut a small hole (less than 10 mm diameter) in the side of the egg, through which the embryo was manipulated, and re-seal with tape, a glass cover slip, shell membrane or a piece of shell. Though relatively simple to perform, this “windowing” method caused embryonic mortality between 70 and 100%. Improved access to the embryo and increased survival and hatchability can be achieved if the embryo is transferred to surrogate eggshells for incubation to hatching using two different shells and a method adapted from published techniques (Callebaut, (1981) Poult. Sci 60: 723-725) and (Rowlett, K. and K. Simkiss, (1989) J. Exp. Biol. 143: 529-536), which are specifically incorporated herein by reference. With this modified technique, the mean hatch rate is approximately 41% (range 23-70%) with 191 chicks hatched from 469 cES-cell injected embryos.

Incubation of embryos following injection of donor ES cells into recipient embryos can be divided into two parts comprising System A and System B as described below:

System A covers the first three days of post-oviposition development. Fertile eggs containing the recipient embryos are matched with eggs 3 to 5 grams heavier. A 32 mm diameter window is cut at the pointed pole, the contents removed and the recipient embryo on the yolk, together with the surrounding albumen, is carefully transferred into the surrogate shell.

Cells are taken up in a sterile, finely tapered glass pipette connected to a mouth aspirator fitted with a 2 micron filter. The opening of the pipette can be from 50 to 120 microns in diameter and possesses a 30° spiked bevel. The embryo is visualized under low magnification and with blue light. Chicken ES cells are trypsinized into a single cell suspension and 4,000 to 26,000 cells are injected into an embryo. The cells are gently expelled into the space either below or above the embryo, i.e. into the sub-embryonic cavity or between the apical surface of the area pellucida and the perivitelline layer (yolk membrane). Extra albumen collected from fresh fertile eggs is added and the shell sealed with Saran Wrap™ plastic film.

System B covers the period from day three to hatching. At day three of incubation the embryo has reached around stage 17 (H&H). Water has been transported from the albumen to the sub-embryonic cavity, causing the yolk to enlarge and become very fragile. The contents of the System A shell are very carefully transferred to a second surrogate shell (usually a turkey egg) 30 to 35 grams heavier than the original egg. Penicillin and streptomycin are added to prevent bacterial contamination and the 38 to 42 mm window in the blunt pole is sealed with plastic film. This larger shell allows for an artificial airspace. At day 18 to 19 of incubation the embryo cultures are transferred to tabletop hatchers for close observation. As lung ventilation becomes established, holes are periodically made in the plastic film to allow ambient air into the airspace. Approximately 6-12 hours before hatching the film is replaced with a small petri dish, which the chick can easily push aside during hatching.

For incubation, conventional temperature (37.5 to 38° C.) and relative humidity (50 to 60%) are maintained for the embryos in surrogate shells, but periodic egg rocking, which is normally hourly and through 90 degrees, has to be modified to ensure good survival. In System A rocking is through 90° every 4 to 5 minutes; in System B it is through 40 to 60° every 40 to 45 minutes. In both systems the speed of rocking is maintained at 15 to 20° per minute.

The contribution of cES cells to chimeras is improved if the recipient embryo is: (1) irradiated by exposure to 660 rads of gamma irradiation, or (2) altered by mechanically removing approximately 1000 cells from the center of the embryo, or by combining (1) and (2) before the injection of the cES cells. Referring to Table 5, contribution of cES cells to the somatic tissues increased substantially when recipient embryos were compromised, either by removing cells from the center of the recipient embryo or by exposure to irradiation. When the recipient embryos are compromised by a combination of irradiation and mechanical removal of the cells, the contribution of the ES cells is increased further, even though the cES cells had been in culture for longer periods of time. Some of the resulting chimeric chicks are indistinguishable from pure Barred Rock chicks. As the data in Table 5 show, chimerism rates as well as the extent of chimerism per embryo increases after compromising the recipient embryo.

TABLE 5
Frequency of somatic chimerism after injection of cES cells into recipient embryos
that were compromised by different methods.

| | | | | | | Extent |
|---|---|---|---|---|---|---|
| | # | Time | | # Embryos & | Frequency of | feather |
| Treatment to compromise | Cell | cells in | # | chicks | chimerism | chimerism |
| the recipient embryo | lines | culture | Chimeras | evaluated | % | (%) |
| None | 14 | 4-106 days | 83 | 347 | 24 | 26 |
| Mechanical removal of cells | 1 | 6 months | 34 | 63 | 54 | 20 |
| Irradiation | 1 | 6-7 months | 56 | 95 | 59 | 29 |
| Irradiation & | 1 | 7-8 months | 52 | 59 | 88 | 49 |
| Mechanical removal of cells | | | | | | |

Recipient embryos substantially younger than stage X may also be used to produce chimeras using ES cell as the donor. Early stage recipient embryos are retrieved by injecting the hens with oxytocin to induce premature oviposition and fertile eggs are retrieved at stages VII to IX.

Alternatively, the retrieval of embryos from the magnum region of the oviduct provides access to stage I to VI embryos, consisting of approximately 4-250 cells, and enables the development of chimeras from all embryonic stages as potential recipient embryos.

Transfection of cES cells may be achieved by lipofection and electroporation. Referring to Table 6, an appropriate amount of DNA compatible with the size of the well being transfected is diluted in media absent of serum or antibiotics. The appropriate volume of Superfect (Stratagene) is added and mixed with the DNA, and the reaction is allowed to occur for 5-10 minutes. The media is removed and the wells to be transfected are washed with a Ca/Mg free salt solution. The appropriate volume of media, which can contain serum and antibiotics, is added to the DNA/superfect mixture. The plates are incubated for 2-3 hours at 37 C. When the incubation is completed, the Superfect is removed by washing the cells 1-2× and fresh culture media is added.

TABLE 6
Conditions for transfection of chicken ES cells using Superfect.

| | Volume | | | | | |
|---|---|---|---|---|---|---|
| | of media | | | Time to | Volume | |
| | used | Total | | form | of media | |
| | to dilute | amount | ul | complex | added to | Incubation |
| Plate Size | DNA | of DNA | Superfect | (min) | complex | time |
| 96 well | 30 ul | 1 | 5 ul | 5-10 | 150 | 2-3 hrs |
| 48 well | 50 ul | 1.5 | 9 ul | 5-10 | 250 ul | 2-3 hrs |
| 24 well | 60 ul | 2 | 10 ul | 5-10 | 350 ul | 2-3 hrs |
| 12 well | 75 ul | 3 | 15 ul | 5-10 | 400 ul | 2-3 hrs |
| 6 well | 100 ul | 4 | 20 ul | 5-10 | 600 ul | 2-3 hrs |
| 60 mm | 150 ul | 10 | 50 ul | 5-10 | 1000 ul | 2-3 hrs |
| 100 mm | 300 ul | 20 | 120 ul | 5-10 | 3000 ul | 2-3 hrs |

A petri-pulser is used to electroporate cES cells that are attached to the plate in a 35 mm diameter well. The media is removed and the well is washed with a salt solution without Ca⁺⁺ and Mg⁺⁺. One ml of electroporation solution is added to the well. DNA is added and the media is gently mixed. The petri-pulser is lowered onto the bottom of the well and an electrical current is delivered. (Voltage preferably varies from 100-500 V/cm and the pulse length can be from 12-16 msec). The petri-pulser is removed and the electroporated well is allowed to stand for 10 minutes at room temperature. After 10 minutes, 2 mls of media is added and the dish is returned to the incubator.

To transfect cells in suspension, media is removed and cells are washed with a Ca/Mg free salt solution. Tryspin with EDTA is added to obtain a single cell suspension. Cells are washed, centrifuged and resuspended in a correctional electroporation buffer solution such as PBS. The ES cell suspension is placed into a sterile cuvette, and DNA added (minimum concentration of 1 mg/ml) to the cell suspension and mixed by pipetting up and down. The cells are electroporated and allowed to sit at room temperature for 10 minutes. Cells are removed from the cuvette and distributed to previously prepared wells/dishes. Cells are placed in an incubator and evaluated or transient transfection 24-48 hours after electroporation. Selection of antibiotic resistant cells may also be started by including an antibiotic such as puromycin in the culture medium.

In a preferred embodiment, the concentration of puromycin required for selecting transfected cells is calculated as a titration kill curve. Titration kill curves for chicken embryonic stem cells are established by exposing cells in culture to puromycin concentrations varying from 0.0 to 1.0 μg/ml for 10 days (Table 7) and neomycin concentrations varying from 0.0 to 200 μg/ml (Table 8). The medium is changed every 2 days and fresh puromycin or neomycin is added. When exposed to a concentration of 0.3 μg/ml puromycin, ES cells were absent from all wells after 3 changes of medium with fresh puromycin over a six day period (see Table 7). Puromycin concentrations of 0.3-1.0 μg/ml are used for selection of the transfected cultures. Neomycin concentrations over 40 μg/ml eliminated all cES cells within 7 days (Table 8).

After 10 days of selection, cES cells colonies are visible and can be picked for further expansion.

TABLE 7
Morphology of cES cells after exposure of various concentrations of puromycin and
different lengths of time (days after addition of puromycin).

| Puromycin | Time under selection (days) | | | | | | | | | |
|---|---|---|---|---|---|---|---|---|---|---|
| conc. (μg/ml) | 1 | 2 | 3 | 4 | 5 | 6 | 7 | 8 | 9 | 10 |
| 0.0 | ES | ES | ES | ES | ES | ES | ES | ES | ES | ES |
| 0.1 | ES | ES | ES | ES | ES | ES | ES | ES | ES | ES |
| 0.2 | ES | ES | ES | ES | ES | ES | ES | ES | ES | ES |
| 0.25 | ES | ES | ES | ES | ES | diff | diff | diff/ | diff/ | diff/ |
| | | | | | | | | gone | gone | gone |
| 0.3 | ES | ES | diff | diff/ | diff/ | gone | gone | gone | gone | gone |
| | | | | gone | gone | | | | | |
| 0.4 | ES | diff | gone | gone | gone | gone | gone | gone | gone | gone |
| 0.5 | diff | gone | gone | gone | gone | gone | gone | gone | gone | gone |
| 0.6 | diff | gone | gone | gone | gone | gone | gone | gone | gone | gone |
| 0.7 | diff | gone | gone | gone | gone | gone | gone | gone | gone | gone |
| 0.8 | gone | gone | gone | gone | gone | gone | gone | gone | gone | gone |

ES: ES cells are present.
diff: ES cells are differentiated.
gone: no morphologically recognizable cells are present

TABLE 8
Morphology of cES cells after exposure of various concentrations of neomycin and
different lengths of time (days after addition of neomycin).

| Neomycin | Time under selection (days) | | | | | | | | | |
|---|---|---|---|---|---|---|---|---|---|---|
| conc. | | | | | | | | | | |
| (μg/m1) | 1 | 2 | 3 | 4 | 5 | 6 | 7 | 8 | 9 | 10 |
| 0.0 | ES | ES | ES | ES | ES | ES | ES | ES | ES | ES |
| 10 | ES | ES | ES | ES | ES | ES | ES | ES | ES | ES |
| 20 | ES | ES | ES | ES | ES | ES | ES | ES | ES | ES |
| 30 | ES | ES | ES | ES | ES | ES | ES/ | ES/ | Diff | Diff/ |
| | | | | | | | Diff | diff | | gone |
| 40 | ES | ES | ES | ES | ES/ | Diff/ | dead | gone | gone | gone |
| | | | | | Diff | dead | | | | |
| 50 | ES | ES | ES | ES/ | ES/ | Diff/ | Dead/g | gone | gone | gone |
| | | | | Diff | Diff | dead | one | | | |
| 60 | ES | ES | ES | gone | gone | gone | gone | gone | gone | gone |
| 100 | ES/ | Diff | dead | gone | gone | gone | gone | gone | gone | gone |
| | Diff | | | | | | | | | |
| 150 | dead | dead | gone | gone | gone | gone | gone | gone | gone | gone |
| 200 | dead | gone | gone | gone | gone | gone | gone | gone | gone | gone |

Selection of transfected chicken ES cells and their identification in chimeras requires that the transgene confer a selective advantage to the cells in culture (e.g. resistance to puromycin in the medium) and that it produce an identifiable gene product in the cells in the chimera which are derived from the ES cells. This can be accomplished using pCX/GFP/Puro which provides resistance to puromycin in cES cells and produces a green fluorescent protein (GFP) in most, if not all, donor-derived cells in chimeras.

Referring to FIG. 1, PCX/GFP/Puro was produced in three cloning steps involving two intermediates before make the final pCX/GFP/Puro plasmid. In step 1, the PGK-driven Puromycin resistant gene cassette (1.5 Kb) was released from pKO SelectPuro (Stratagene) by Asc I digestion. The fragment was then blunted and Kpn I linkers were added. The resulting fragment (GFP/Puro) was inserted into the corresponding Kpn I site of pMIEM (courtesy of Jim Petitte (NCSU), a GFP expression version derived from LacZ expression pMIWZ, see Cell Diff and Dev. 29: 181-186 (1990) to produce the first intermediate (pGFP/Puro). The PGK-Puro cassette was in same transcription orientation as GFP (determined by BamH I and Sty I digestion). In step 2, the GFP/Puro expression cassette (2.5 Kb) was released from pGFP/Puro by BamH I and EcoR I double digestion. The resulting fragment was inserted into the BamH I and EcoR I sites of pUC18 (Invitrogen). It contains 5′ unique sites, Hind III, Pst I and Sal I. The resulting plasmid pUC18/GFP/Puro was verified by a BamH I, EcoR I, and Not I triple digestion. In the third step, the CX promoter including 384 bp CMV-IE enhancer, 1.3 kb chicken beta-actin promoter and portion of 1^{st} intron was released from pCX-EGFP (Masahiro, 1. et al., (1995) FIBS Letters 375: 125-128) by Sal I and EcoR I digestion. A 3′ EcoR I (null)-Xmn I-BamH I linker was attached to the fragment and it was inserted into the Sal I and BamH I sites of pUC18/GFP/Puro. The plasmid pCX/GFP/Puro was verified by a BamH I and Pst I double digestion. pCX/GFP/Puro DNA can be linearized by Sca I digestion for transfection into cES cells.

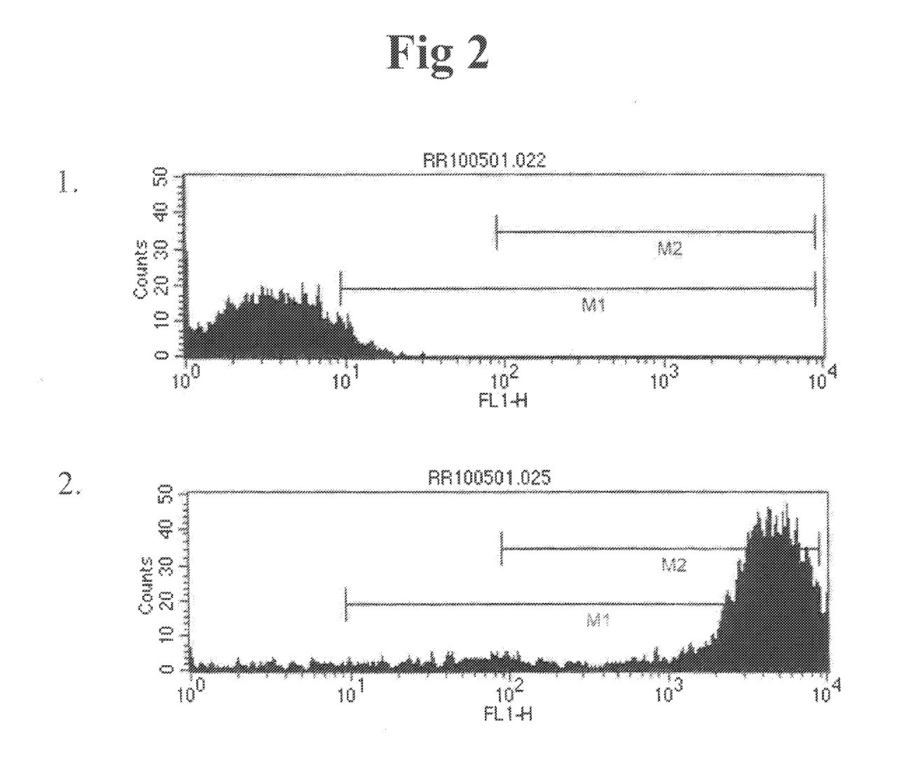

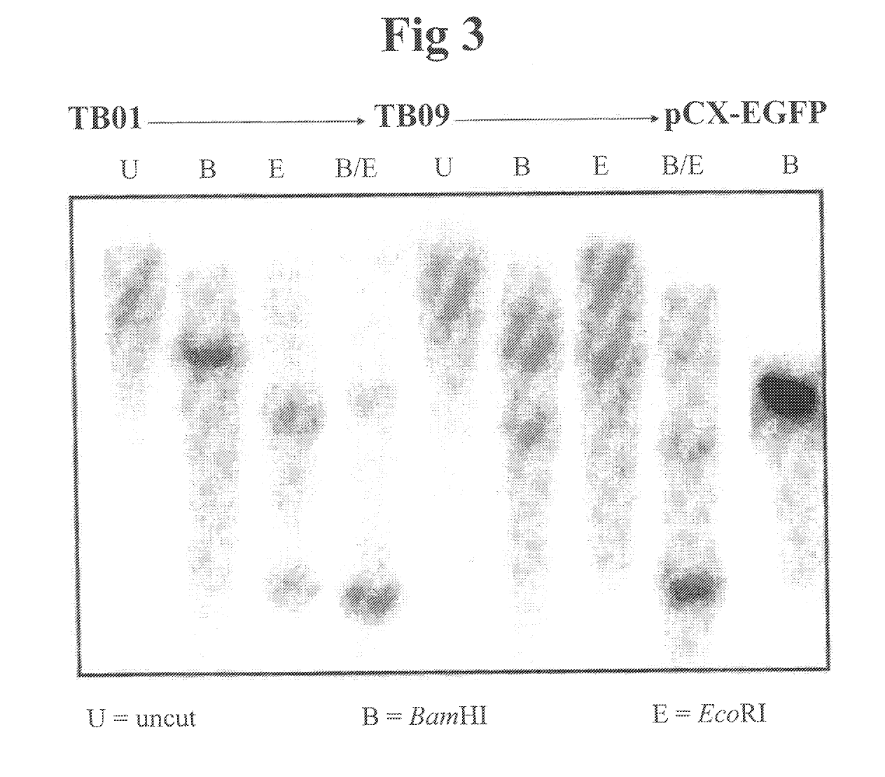

Transfection and selection of ES cells using the procedures described above produced a population of cells that would grow in the presence of 0.5 ug of puromycin. These cells exhibited green fluorescence when examined by conventional fluorescence microscopy. When preparations of the ES cells are examined by fluorescence activated cell sorting, it is evident that essentially all of the cells carry and express the transgene (See FIG. 2). Southern analysis of DNA from the transfected ES cell lines TB01 and TB09 that was digested with BamHI, EcoRI or both restriction endonucleases revealed the transgene in DNA fragments of various sizes, providing evidence that the transgene is integrated into the genome (See FIG. 3).

The CX/GFP/Puro construct demonstrates that transgenes of at least 4.5 kb can be inserted into chimeric chickens. Using the cES cells described herein, chicken ES cells can be transfected with different or larger constructs. A very large transgene encoding part of the unrearranged human heavy chain immunoglobulin locus has been transfected into chicken ES cells. A 139 kilobase bacterial artificial chromosome (BAC) clone was co-transfected with the pCX-EGFP-CX-puro selectable marker into cES cells by co-lipofection of circular BAC DNA and linear selectable marker DNA. The BAC clone contains a human genomic DNA insert from an unrearranged immunoglobulin heavy chain locus and contains the most 3′ variable region (V_{H}6-1), all the diversity (D) segments, all the joining (J) segments, the Cmμ and Cγ constant regions, the J-intronic enhancer, and all the intervening DNA between these elements. It also includes the human gene KIAA0125, a gene that encodes a non-translated RNA of unknown function that is found between V_{H}6-1 and the D segment region. pCX-EGFP-CX-puro is a plasmid that contains the Enhanced Green Fluorescent Protein (EGFP) gene driven by the CX promoter (consisting of a cytomegalovirus enhancer and the chicken β-actin promoter) and a puromycin resistance gene driven by the same promoter. The cES cells transfected with this plasmid are green fluorescent and resistant to the antibiotic puromycin. The presence of the unrearranged human heavy chain locus in the transfected ES cells that were growing in the presence of puromycin was examined by PCR analysis of transgenes spread throughout the 139 kb construct. The primer sequences used in the PCR analysis were:

V_{H}6-1:
SEQ ID NO. 1
V6-1F AGTGTCAGGGAGATGCCGTATTCA
SEQ ID NO. 2
V6-1R ACTTCCCCTCACTGTGTCTCTFG
D1-26:
SEQ ID NO. 3
D1-26F GGGCGCCTGGGTGGATTCTGA
SEQ ID NO. 4
D1-26R GTGGCCCCTAAACCTGAGTCTGCT
D1-20:
SEQ ID NO. 5
D1-20F CCCGAGCACCGTCCCCATTGA
SEQ ID NO. 6
D1-20R GTGCCGGTGATCCCTGTCTTTCTG
C□:
SEQ ID NO. 7
Mu1F GCGGGAGTCGGCCACCATCACG
SEQ ID NO. 8
Mu1R AGCACAGCCGCCGCCCCAGTAG
C□□
SEQ ID NO. 9
Delta1F TGGGGAGAGGAGAGCACAGT
SEQ ID NO. 10
Delta1R GGCGGGCGTAGGGGTCAGC

cES cells are co-transfected with the selectable marker pCX-EGFP-CX-puro and the BAC CTD-2005N2, resulting in a cES cell line designated BAC-A. Genomic DNA is prepared and PCR performed using 5 different primer sets corresponding to markers along the length of the BAC clone. These markers are: V_{H}6-1 (24 kb from the 5′ end [relative to the human heavy chain locus] of the human genomic insert), D1-26 (83 kb from the end), D1-20 (73 kb from the end), Cμ (˜108 kb from the end), and Cδ (˜120 kb from the end). Only V_{H}6-1, Cμ, and D1-26 are shown but all gave similar results. As a control for amplification from the cES cell sample, a chicken β-actin PCR is also run. The samples are:
1. BAC-A cells;
2. Mouse STO cells used as a feeder layer for the cES cells (negative control);
3. Barred Rock embryo DNA (the same strain as the parental cES cell line, negative control);
4. Human genomic DNA (positive control);
5. cES cell medium (negative control).

All segments of the transgene are present in the transfected and selected ES cells.

As noted above, genetic modifications that produce modified avian chromosomes are preferably conducted in a cell line which exhibits high frequency homologous recombination. These recombination proficient cell lines are particularly useful for performing genetic modifications and creating genetic constructs that are then used in transgenesis. A preferred example is the DT40 avian pre-B cell line. DT40 cells are highly efficient in gene targeting recombination events and have been used to modify mammalian genetic loci to study gene expression and regulation. The use of DT40 cells to efficiently modify human chromosomal genes is well known and has been described for the production of modified human chromosomes. Dieken et al. transferred individual human chromosomes into the avian DT40 cell line by microcell fusion, achieved efficient systematic manipulation of human chromosomes, and then returned the human chromosome to human cell line with appropriate gene expression. The improved rate of homologous recombination can be utilized to obtain selective gene targeting. Dieken et al., (1996) Nature Genetics, Vol. 12:174-182. The generation and maintenance of DT40 cells with mammalian chromosome(s) is described in U.S. Pat. No. 5,543,319 which is specifically incorporated by reference herein.

Once a modified avian chromosome comprising human immunoglobulin loci has been constructed, the chromosome must be transferred from one cell to a pluripotent cell for injection into a recipient embryo. The pluripotent cell is preferably an avian embryonic stem cell and may also be comprised of specific genetic modifications, such as the functional disruption of immunoglobulin loci as described herein. The introduction of limited numbers of chromosomes from one cell to another is well established in the literature. One method uses small cell-like structures, termed microcells, containing a limited amount of genetic material within a micronucleus that is itself surrounded by a rim of cytoplasm and an intact plasma membrane. Microcells provide an efficient vector for transferring chromosomes into recipient cells using cell fusion methods. The donor chromosomes are typically marked to facilitate selective retention of specific donor chromosome(s) in the resulting microcell hybrids. Suitable selectable markers for mammalian and avian cells are well known and include the E. coli gpt gene (confers xanthine-aminopterin-thymidine resistance), the neomycin resistance gene (neo) from transposon Tn5 (confers G418 resistance), the hygromycin phosphotransferase gene (confers resistance to hygromycin B), the herpes simplex virus type I (HSV) thymidine kinase (tk) gene (complements TK⁻ cells and confers hypoxanthine-aminopterin-thymidine (HAT) resistance), the hypoxanthine phoshoribosyltransferase (HPRT) gene (complements HPRT⁻ cells and confers HAT resistance), and the adenine phosphoribosyltransferase (APRT) gene (complements APRT⁻ cells and confers adenine-aminopterin-thymidine (AAT) resistance).

Chromosomes may be marked with selectable markers by integration in a number of well established ways (see, for example, Fournier and Frelinger, (1982) Mol. Cell. Biol. 2:526-534; Athwal et al., (1985) Som. Cell. Mol. Genet. 11:177-187; Saxon et al., (1985) Mol. Cell. Biol. 5:140-146; Tunnacliffe et al., (1983) EMBO J. 9:1577-1584; and Lugo et al., (1987) Mol. Cell. Biol. 7:2814-2820, which are incorporated by reference herein in their entirety). These methods rely on random integration events to insert marker genes into the chromosomes of the host cell. Certain of these methods, such as defined translocations (Fournier and Frelinger, ibid.) and calcium phosphate mediated coprecipitation (Saxon et al., ibid.), are limited due to a low number of suitable translocations or a low frequency of gene transfer, respectively. Alternatively, plasmids containing expression units capable of directing the expression of selectable marker genes may be electroporated into cells using, for example, a Bio-Rad Gene Pulser™ apparatus to transfect linear plasmids into suitable recipient cells. One suitable vector for this purpose is the tgCMV/HyTk plasmid (Lupton et al., Mol. Cell. Biol. 7:2814-2820, 1987; which is incorporated by reference herein in its entirety) which confers hygromycin resistance to the recipient cell. The plasmid also complements TK⁻ cells. Plasmid tgCMV/HyTk contains an in-frame fusion between the hygromycin phosphotransferase gene and the herpes simplex virus type I thymidine kinase under the control of the human cytomegalovirus IE4 promoter.

### Example 1

### The Functional Disruption or Knockout of the Endogenous Avian Immunoglobulin Gene by Homologous Recombination in Avian Embryonic Stem Cells

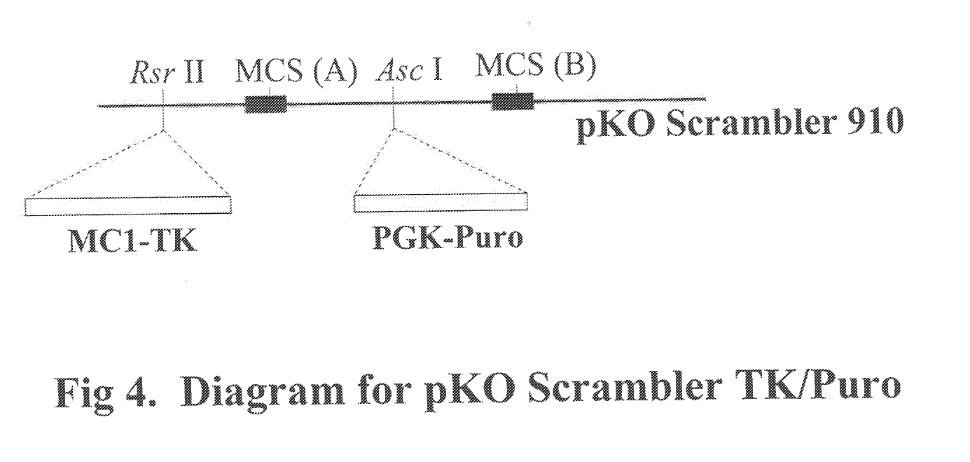

The puromycin expression cassette (1.5 Kb) was released from pKO SelectPuro (Stratagene) by Asc I digestion. Referring to FIG. 4, the resulting fragment was inserted into the Asc I site of pKO Scrambler 910 (Strategene), and verified by a Xho I digestion. Thymidine Kinase expression cassette (2.0 Kb) was released from pKO SelectTK (Stratagene) by Rsr II digestion. The resulting fragment was inserted into the Rsr II site of pKO Scrambler Puro, and verified by Sph I digest. The plasmid illustrated in FIG. 4 is the starting point for all the IgH and IgL targeting constructs.

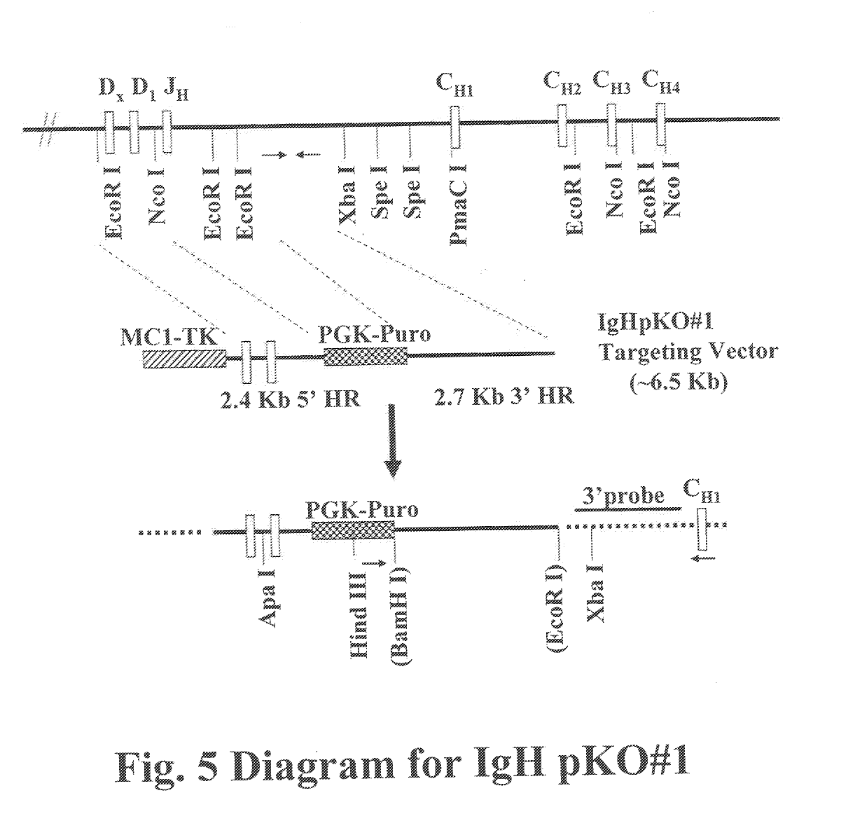

A genomic DNA fragment of chicken IgH (DJ-6) in germline configuration was obtained from Dr. Claude-Agnes Reynaud, University Paris, France. The 6.2 Kb EcoR I fragment contains coding sequences of the chicken IgH D_{X}, D_{I}, and J_{H}. Referring to FIG. 5, the D_{X}, D_{I}, and J_{H} region of the chicken IgH (6.2 kb) was released from the vector portion of the DJ-6 plasmid by EcoR I. A subsequence Nco I partial digest of this fragment isolated a 2.2 Kb DNA fragment containing D_{X} and D_{I}. The fragment was blunted and inserted into Hpa I site (Scrambler A region) of pKO Scrambler TK/Puro to become the 5′ homologous region. This intermediate plasmid pKO TK/Puro-D_{X}-D_{I} was verified with a Nco I digestion.

A 2687 bp fragment of chicken IgH switch and constant region (base 11-2697, Genebank #AB029075) was amplified from chicken genomic DNA. Primers Cu-1 (with BamH I site underlined) and Cu-2 (with EcoR I site underlined) were designed based on the above referenced sequence.

(SEQ ID NO. 11)
Cu-1: 5′-CTCGGATCCCAACAAACGGCACTCGATAATT-3′
(SEQ ID NO. 12)
Cu-2: 5′-CTCGAATTCTTCATTGACCTTCATTAACCGC-3′

The PCR product was cloned into pGEM-T easy vector (Promega) to form Cu2.7. The plasmid was confirmed by sequencing from the two ends.

The 2.7 Kb fragment of chicken IgH switch and constant region was released from Cu2.7 with BamH I and EcoR I. This fragment was inserted into the BamH I and EcoR (Scrambler B region) of pKO TK/Puro-D_{X}-D_{I} to form the 3′ homologous region. The resulting plasmid IgHpKO#1 (see FIG. 2) was verified by a BamH I and EcoR I double digestion. The clone was confirmed by sequencing from two ends for the presence of chicken IgH sequences. This plasmid can be linearized with Not I, Sal I or Sca I. After purification, the linearized DNA is ready for transfection.

A genomic DNA fragment of chicken IgL (Cλ36SacI) in germline configuration was a gift from Dr. Claude-Agnes Reynaud, University Paris (EMBO J. (1993) 12:4615-23). The 10.5 Kb Sac I fragment contains coding sequences of chicken IgL V_{L}, J_{L}, and C_{L}, and 2.0 Kb and 3.8 Kb of 5′ and 3′ flanking sequences, respectively. Two IgL KO constructs are made.

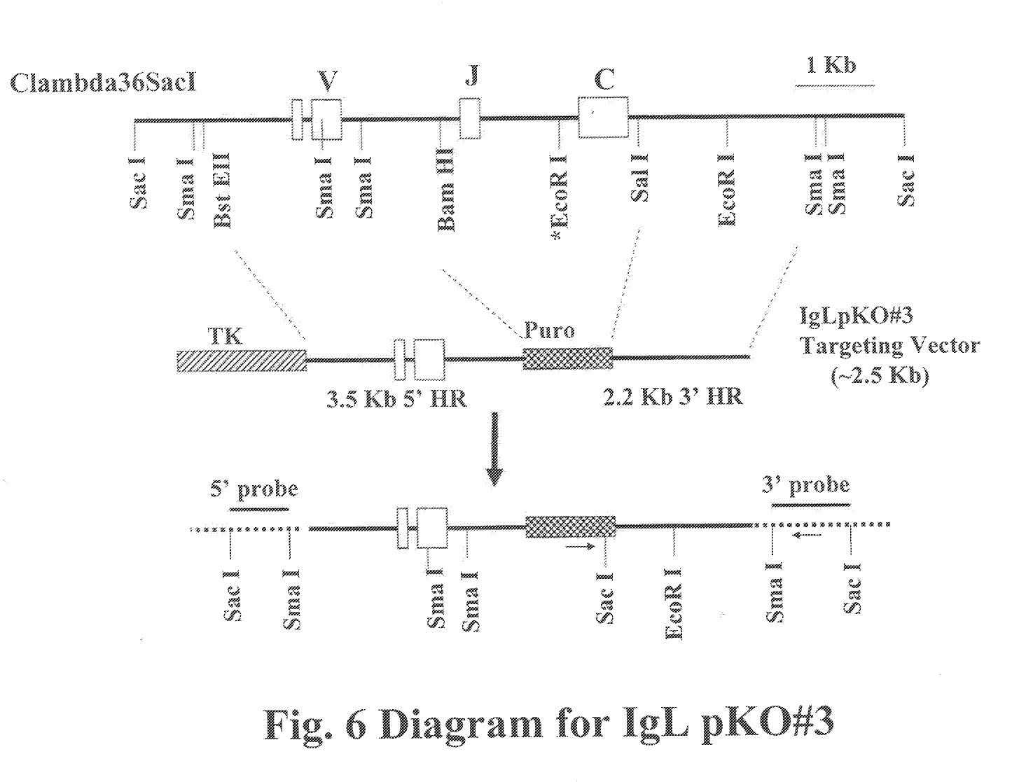

Referring to FIG. 6, a 2.0 Kb fragment of chicken IgL 3′ flanking sequences was released from Cλ36SacI by Sal I and Sma I double digestion. The fragment was then blunted and inserted into the Sma I site (Scrambler B region) of pKO scrambler TK/Puro to form the 3′ homologous region. This intermediate plasmid 3′IgLpKO-TK/puro was verified with EcoR I digestion for correct orientation.

The chicken IgL fragment (10.5 Kb) was released from Cλ36SacI by Sac I digestion. A subsequent BstE II and BamH I double digestion isolated the fragment of IgL V region (3.5 Kb). The fragment was then blunted and inserted into the Hpa I site (Scrambler A region) of the 3′IgLpKO-TK/Puro to become the 5′ homologous region. This plasmid IgLpKO#3 (see FIG. 6) was verified with Sma I and Rsr II double digestion for correct orientation. The plasmid was further confirmed by sequencing from the two ends. The plasmid can be linearized with Sal I and purified for transfection.

In another embodiment, a 2396 bp fragment of chicken IgL V region (base 24-2419, Genebank #M24403) was amplified from chicken genomic DNA. Primers CiGL5A (with Hpa I sequence underlined) and CiGl5B (with Hpa I sequence underlined) were designed based on the above-referenced sequence.

(SEQ ID NO. 13)
cIgL5A: 5′-CTCGTTAACGATGTTGTACTGAGGGATGTGG-3′
(SEQ ID NO. 14)
cIgL5B: 5′-CTCGTTAACCGGTGAACAAGGATGTTCAGTA-3′

The PCR product was cloned into pGEM-T easy vector (Promega). The resulting plasmid 5′IgL was confirmed by sequencing from the two ends. The 2.4 Kb V_{L} region of chicken IgL was released from 5′ IgL plasmid by Hpa I digestion. The fragment was then inserted into Hpa I site (Scrambler A region) of pKO scrambler TK/Puro. This intermediate plasmid 5′IgLpKO-TK/Puro was verified with a KpnI or NsiI digestion for correct orientation.

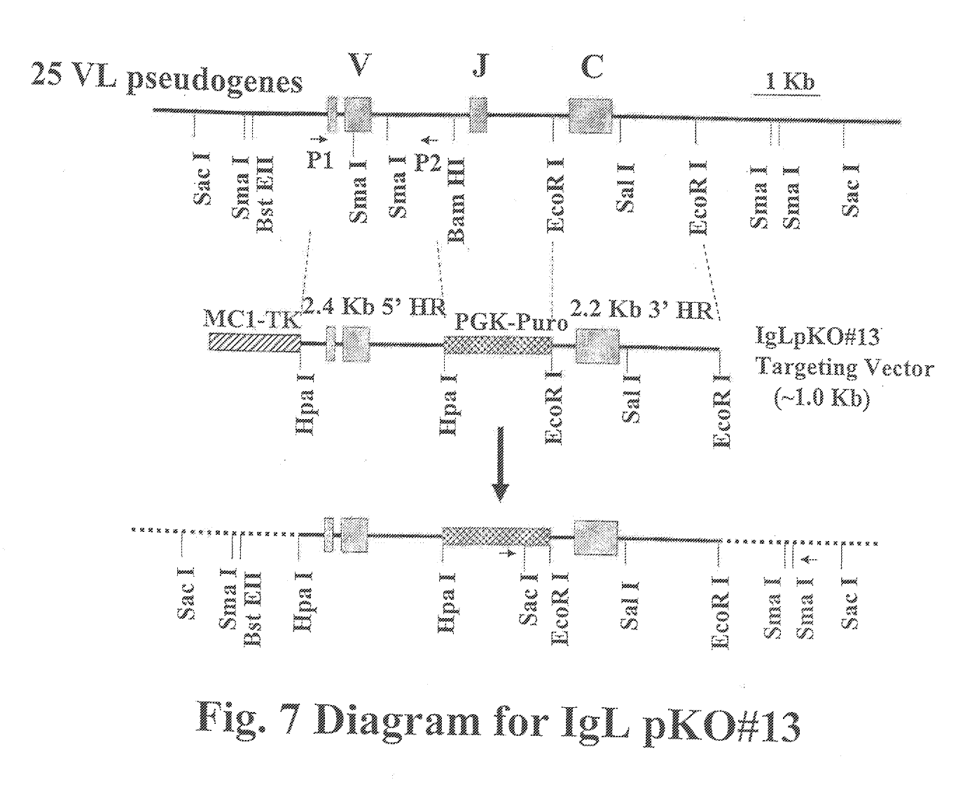

A 2.8 Kb fragment of chicken IgL C region was released from Cλ36SacI by EcoR I digestion. The fragment was then inserted into the EcoR I site (Scrambler B region) of 5′IgLpKO-TK/Puro to form the 3′ homologous region. This plasmid IgLpKO#13 (see FIG. 7) was verified with Nsi I digestion for correct orientation and further confirmed by sequencing from the two ends. IgLpKO#13 can be linearized with Not I and purified for transfection.

### Example 2

### MMCT with DT40 Cells as Chromosome Donors

DT40 cells containing chromosome of interest, such as an avian chromosome 15 lacking the immunoglobulin heavy chain locus, are grown up in DMEM/10% FBS/5% chicken serum/10% tryptone phosphate broth/0.1 μm β-mercaptoethanol/2 mM glutamine/pen-strep and appropriate selection drug. 1.6×10⁸ cells are obtained and demecolcine is added to 0.01 μg/ml final concentration (1:1000) and maintained for 48-72 hours. Fresh Percoll (Pharmacia) is prepared by equilibrating with NaCl to a final concentration of 150 mM and Hepes buffer, pH 7.0, to a final concentration of 50 mM. 17.5 ml of equilibrated Percoll is added to 6 50-ml Oak Ridge polycarbonate tubes (Nalgene). DT40 cells are harvested by pelleting (save 500 μl for Hoechst staining). The cell population is resuspended in 105 ml DMEM/10% FBS/20 μg/ml cytochalasin B (1.3×106 cells/ml) and cell clumps are broken up by trituration before loading onto the gradient. 210 μl of Demecolcine are added to cells before combining with Percoll for a final concentration of 0.01 μg/ml and 17.5 mls of cells are added to each Percoll tube and mixed well by inverting. The resulting composition is centrifuged in an Avanti centrifuge with a JA-25.50 rotor at 19,415 rpm (30,924 g) for 80 min at 32° C. (no brake). Material is pooled from about 2 cm below the top of each tube to the region just above the Percoll pellet and centrifuged again at 2000 g for 5 minutes. The microcells are resuspended in 50 ml DMEM (no serum) by vigorous pipetting. This step is repeated a total of 3 times to rid the suspension of all Percoll. The cells are filtered sequentially through 8 μm, then 5 μm, then 3 μm filters yielding 3-9×10⁷ microcells. An aliquot of 500 μl was saved for Hoechst staining. 10⁷ recipient cells are harvested, washed three times with DMEM (no serum) and resuspended in 5 ml DMEM, prior to being combined with microcells and centrifuged at 1250 rpm for 5 minutes to remove supernatant. The cells and microcells are resuspended in 5 ml DMEM+100 μg/ml phytohemagglutinin P for 10 minutes at room temp, and then spun down. The pellet is dispersed by tapping and then slowly dripping 0.3 ml of a PEG solution (0.25 g sterile PEG in glass vial, melt, add 50 μl DMSO, 0.3 ml DMEM). One ml DMEM is immediately added in a dropwise fashion while swirling, then another 1 ml, then 7 ml with gentle swirling. The resulting mixture is centrifuged at 1000 rpm for 5 minutes, rinsed in DMEM, and re-centrifuged. The pellet is resuspended in regular growth media and plated. After 24 hours the media is replaced with selective media.

### Example 3

### The Functional Disruption or Knockout of the Endogenous Avian Immunoglobulin Heavy Chain Gene by Telomere-Associated Chromosomal Locus Deletion

In this embodiment of the invention, a construct comprising a telomere is used as a construct to eliminate the endogenous avian immunoglobulin heavy chain gene. In a preferred embodiment, the construct is comprised of a human immunoglobulin locus and an homologous region to avian chromosome 15 is used as a targeting construct to delete the avian immunoglobulin heavy chain locus. The construct is designed to accomplish the complete deletion of the avian immunoglobulin heavy chain locus and the construction of a chimeric chromosome containing avian DNA and a telomeric region comprising an unrearranged human immunoglobulin locus. Following recombination of this construct and an avian embryonic stem cell, embryonic stem cells with the deleted avian immunoglobulin locus can be selected and used to generate chimeric birds having functionally disrupted endogenous immunoglobulin loci and featuring expression of a human immunoglobulin locus upon exposure to an antigen. For the purposes of this embodiment, the construct includes a telomeric fragment, a selectable marker, a functionally unrearranged human immunoglobulin locus, and a region of homology with avian chromosome 15. In this embodiment, the orientation of the immunoglobulin locus is such that the entire portion of the functional locus is located in a region that is proximate to the telomere of the chromosome. Successful homologous integration events are identified by PCR and southern blotting. Embryonic stem cells demonstrating successful integration of the construct described above are injected into embryos, cultured until birth, and analyzed for successful incorporation of the construct.

### Example 4

### Construction of Modified Avian Chromosome 15

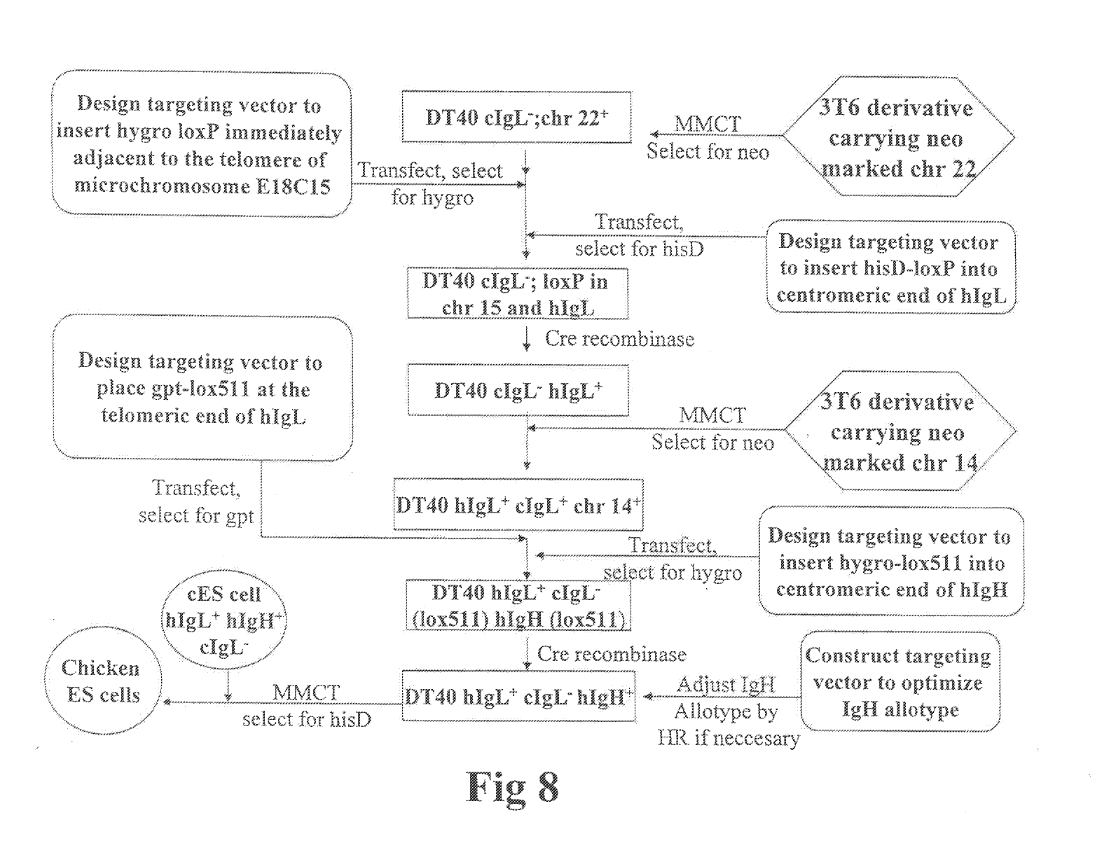

Referring to FIG. 8, the construction of the engineered chromosome of the invention includes the insertion of a recombination site such as a Lox site centromeric of a region of DNA comprised of the endogenous immunoglobulin heavy chain gene or directly within a recombination-competent site such as the J region. In a preferred version of this embodiment, at least one site-specific recombination site is inserted at a point such that deletion of all of the DNA telomeric of the site renders the chicken immunoglobulin heavy chain locus non-functional or non-existent. Subsequently, a construct containing a complimentary recombination site attached to a segment of exogenous DNA comprised of at least one human immunoglobulin locus is inserted into the cell. Preferably, the “construct” in this context is the entire human chromosome 2, 14, or 22 containing a recombination site centromeric of the human heavy or light chain immunoglobulin gene respectively. When the construct is introduced into the DT40 cell under conditions that facilitate site-specific recombination, the human immunoglobulin locus replaces the avian immunoglobulin heavy chain locus via recombination of the Lox site. Thus, when the modified avian chromosome containing the Lox recombination site and the human chromosome with an immunoglobulin locus and a complementary Lox site are combined in the DT40 cell under conditions causing recombination, the construct replaces all of the endogenous DNA that is telomeric of the recombination site and the endogenous chicken immunoglobulin heavy chain gene is deleted and replaced with a construct containing human immunoglobulin loci.

The first construct, when integrated into the avian chromosome may also contain a second recombination site that is telomeric of the unrearranged human immunoglobulin locus. In this embodiment, at least two human immunoglobulin loci may be inserted into the modified avian chromosome 15. Because the human immunoglobulin heavy chain locus is known to be telomeric at chromosome 14, it is preferred that the deletion or functional disruption of the avian immunoglobulin heavy chain locus be achieved with a construct comprised of the human immunoglobulin light chain locus as a first step. Thus, the first construct is human chromosome 14 with an appropriate recombination site. When a first recombination step occurs, the modified chromosome is comprised of the native avian chromosome 15 absent the immunoglobulin heavy chain locus, but having the human immunoglobulin light chain locus at the telomeric end. As a second stage, a dissimilar recombination site, such as a second Lox site is inserted at the telomeric end of the human immunoglobulin light chain locus that is now part of the modified avian chromosome.

Alternatively, two dissimilar recombinations sites, such as a Lox P and Lox 511 site, can be simultaneously inserted into human chromosome 14 such that the first recombination step provides two dissimilar recombination sites. By either approach, the chimeric chromosome has a second recombination site at a telomeric end. Thus, in this intermediate configuration, the modified, engineered avian chromosome 15 contains a first recombination site centromeric of the human immunoglobulin light chain locus and a second dissimilar recombination site telomeric of the locus. This configuration is suited for reaction with a second construct containing the portion of DNA from human chromosome 14 comprising the human immunoglobulin heavy chain locus. Placed under conditions suitable for recombination of the second (but not the first) recombination site, the human immunoglobulin heavy chain locus is integrated into avian chromosome 15 at a site telomeric of the human immunoglobulin light chain lamda locus. In a similar fashion, a second human immunoglobulin light chain locus may be integrated into avian chromosome 15 in an orientation compatible with the existing loci locus.

The modified avian chromosome of the invention has several advantages and unique features compared to existing avian chromosomes and other genetic modifications that have been made for the production of human immunoglobulins. In the configuration described above, the modified chicken chromosome 15 is capable of expressing human immunoglobulins from an avian chromosome. Moreover, in the preferred embodiment, the modified avian chromosome expresses both heavy and light chains of the human immunoglobulin repertoire. Thus, contrary to the endogenous human immunoglobulin gene loci, both chains are expressed from loci on the same chromosome. Furthermore, the modified avian chromosome contains human immunoglobulin DNA that is both integral to the avian chromosome and is oriented in germline configuration and, therefore exists in an unrearranged state that, when successfully used in a transgenic application, results in human immunoglobulin DNA integral to the avian chromosome that is capable of responding to antigen challenge by rearranging to encode immunoglobulin molecules specific for the antigen. Moreover, the deletion of the entire endogenous chicken heavy chain locus avoids the potential for trans-chromosomal switching sometimes observed in murine transgenic immunoglobulin production models. Because the heavy chain immunoglobulin gene disruption in murine models is a deletion of a recombination-competent locus such as a J segment, that prevents immunoglobulin production by preventing V-D-J joining prior to combination of the V-D-J light chain subassembly with an immunoglobulin heavy chain, the endogenous murine constant region DNA remains in place even in the knockout animal. In the mouse model, the remaining murine constant region is available to join with a rearranged V-D-J subunit of the exogenous human DNA, thus resulting in a chimeric antibody that is partially human and partially murine. The strategy described above eliminates this possibility by deleting the endogenous heavy chain immunoglobulin locus.

### Example 5

### Chromosome Transfer

When a modified chromosome is assembled as described above, the chromosome is transferred to a pluripotent cell such as an ES cell. The methods used to transfer chromosomes to pluripotent cells include chromosome-mediated and microcell-mediated transfer techniques. Microcells are produced from donor cells (preferably from primary human diploid fibroblasts that have selectable marker genes integrated into the chromosomes) by first exposing the cells to high concentrations of a mitotic inhibitor, such as colcemid for between 24 and 48 hours, at a concentration between 01 and 10 μ/ml. Exposure to the colcemid induces the cells to form micronuclei. The size of the micronuclei will determine the amount of genetic information available for transfer during microcell hybridization. After micronuclei have formed, the cells are enucleated by centrifugation in the presence of 5 to 20 μg/ml cytochalasin B in an appropriately buffered solution such as serum-free growth medium of phosphate-buffered saline (PBS). Enucleation of adherent cells is achieved by centrifuging the cells grown on a solid support, such that the supports are positioned vertically in centrifuge tubes containing the cytochalasin B solution. Non-adherent cells are enucleated by centrifugation through a percoll gradient containing cytochalasin B, as described in more detail below. The micronuclei are recovered from the resulting pellet. The micronuclei are preferably size selected to remove whole cells and to isolate micronuclei that contain approximately one chromosome. Size selection may be accomplished using sequential filtration through 8- and 5-μm filters (Shapero, Langston and Fournier, (1994) Som. Cell. Mol. Gen. 20:215-231; which is incorporated by reference herein in its entirety). Alternatively, micronuclei can be size separated by unit gravity sedimentation on a linear 1-3% bovine serum albumin gradient, taking the upper fraction containing the smaller micronuclei as described by Fournier and Ruddle (1977, Proc. Natl. Acad. Sci. 74:319-323).

In instances in which the artificial engineered avian chromosome is transferred from the recombination proficient avian cell into pluripotent cells, such as avian embryonic stem cells, suitable cells may be obtained by the method of Petitte et al., U.S. Pat. No. 5,565,479 (specifically incorporated by reference). To fuse the microcells with the recipient cells, the preparation of microcells is incubated with the recipient cells for 10 to 15 minutes at 37° C. In the case of adherent cells, the microcells are preferably suspended in a solution of 100-200 mu·g/ml of phytohemagglutinin P and applied to monolayers of recipient cells to allow for agglutination. In the case of non-adherent recipient cells, the microcells and recipient cells are suspended together in a test tube. The microcells are fused to the recipient cells by a sixty second exposure of between 44 to 50% (wt/wt) polyethylene glycol (mW 1300-1600). The microcell hybrids are allowed to incubate overnight in nonselective medium. The cells are then placed under selection in the appropriate medium to select for the presence of cells containing chromosome(s) from the donor cell having an integrated selectable marker gene.

The presence and identification of donor chromosomes in the microcell hybrids may be carried out using any number of well established methods. In the case of microcell hybrids containing human chromosomes, human chromosomes may be detected by, for example, filter hybridization to detect human alleles or DNA markers. Representative human alleles include argininosuccinate synthetase, adenosine deaminase, nucleoside phosphorylase, and insulin, and representative arbitrary DNA markers include G8, 3.6/1.2, pAW101, p267, D20S2, and D7S8 (Lugo et al., ibid.). FISH karyotyping (Trask et al., (1991) Am. J. Hum. Genet. 48:1-15, and Brandiff et al., (1991) Genomics 10:75-82; which are incorporated herein in their entirety) may be used to identify the presence of donor genomic DNA.

### Example 6

### Bacterial Artificial Chromosome (BAC) for Construction of Human Immunoglobulin Loci

To make transgenic chickens expressing human immunoglobulins, BAC clones from the human heavy chain and kappa light chain loci are first transfected into cES cells. For the heavy chain, two BACs are required: one that contains variable gene segments and one that contains D's, J's and constant regions. A choice of variable region BAC exists from any area of the ˜800 kb region upstream of the D region to the end the 3′ end of the V region that contains a number of functional V's, which are selected from available BACs such as RP11-32914; RP11-408N15, RP11-965B13 and others.

For example, RP11-32914 contains VH6-1, VH3-7 and VH6-4, which can be identified with the following primer pairs:

Primer pair for VH6-1:
(SEQ ID NO. 1)
V6-1F AGTGTCAGGGAGATGCCGTATTCA
(SEQ ID NO. 2)
V6-1R ACTTCCCCTCACTGTGTCTCTTG
Primer pair for VH3-7:
(SEQ ID NO. 15)
Vh3-7F GGCTGAGCTGGGTTTTCCTTGTT
(SEQ ID NO. 16)
Vh3-7R CTGTCGCCCCCTGGTGGTC
Primer pair for VH4-4:
(SEQ ID NO. 17)
Vh4-4F CCTGCACAAGAACATGAAACACCT
(SEQ ID NO. 18)
Vh4-4R GACCCGGCCTCTTGCTCTG

The other BAC used in conjunction with the V region BAC contains a majority of the D segments, all the J segments, the J-mu enhancer, and the constant regions mu, delta, gamma3 and gamma1. This BAC (RP11-417P24) spans the D-gamma1 region in germline configuration and thus contains a wild type complete delta-gamma3 interval. This BAC has been sequenced in the course of the Human Genome Project and can be identified using the following sets of PCR primer pairs:

Amplifies a region near D1-26:
(SEQ ID NO. 3)
D1-26F GGGCGCCTGGGTGGATTCTGA
(SEQ ID NO. 4)
D1-26R GTGGCCCCTAAACCTGAGTCTGCT
Amplifies a region near D1-20:
(SEQ ID NO. 5)
D1-20F CCCGAGCACCGTCCCCATTGA
(SEQ ID NO. 6)
D1-20R GTGCCGGTGATCCCTGTCTTTCTG
Amplifies part of the Cmμ constant region:
(SEQ ID NO. 7)
Mu1F GCGGGAGTCGGCCACCATCACG
(SEQ ID NO. 8)
Mu1R AGCACAGCCGCCGCCCCAGTAG
Amplifies part of the Cδ constant region:
(SEQ ID NO. 9)
Delta1F TGGGGAGAGGAGAGCACAGT
(SEQ ID NO. 10)
Delta1R GGCGGGCGTAGGGGTCAGC
Amplifies a part of the delta-gamma3 interval:
(SEQ ID NO. 19)
UncloF GCTGTTGGCCTTTATTTTCTATTG
(SEQ ID NO. 20)
UncloR ATTTGCACCATTTCCTGAGTTG
Amplifies another part of the delta-gamma3
interval:
(SEQ ID NO. 21)
Unclo2F GTGGGTGATAGAATTTGGTGTTTG
(SEQ ID NO. 22)
Unclo2R GTGGTGGGCAGGATGGGATGAT

A single BAC, such as CTD-2005N2, spanning the region from the 3′ most V, VH6-1, through the delta constant region, can also be transfected into cES cells for the purpose of expression of a primary repertoire consisting of IgM and IgD only.

These BACs are either co-transfected with selectable markers or retrofitted with covalently linked selectable markers for positive selection in ES cells. The markers confer puromycin or G418 resistance, and are under the transcriptional control of the CX promoter, which consists of a 384 bp CMV enhancer and a 1.3 kb promoter and first intron from the chicken β-actin gene.

For the kappa light chain, a single BAC containing several VK genes, all 5 Jk's, Ckappa constant region and enhancers is transfected into ES cells. Such BACs are RP11-601N4 (with 2 variable region genes present, VK5-2 and VK4-1), RP11-1134E24 (with 4 VK's, VK1-6, VK1-5, VK5-2, and VK4-1), and RP11-15J7 (with the same complement of kappa light chain elements as RP 11-1134E24). These BACs can be identified with by PCR with the following sets of PCR primer pairs:

Amplifies part of the Jk region:
(SEQ ID NO. 23)
JkappaF ATGCCAGGGACTCTAACAAACTTC
(SEQ ID NO. 24)
JkappaR TTCCCCTCAACAAAAACCTCTC
Amplifies a part of the Ck constant region:
(SEQ ID NO. 25)
CkappaF AGCTCGCCCGTCACAAAGA
(SEQ ID NO. 26)
CkappaR AGGGGAAAACAAGGAAGCAAGTC
Amplifies part of the Vk4-1 variable gene:
(SEQ ID NO. 27)
Vk4-1F GAGAGGGCCACCATCAACTG
(SEQ ID NO. 28)
Vk4-1R AACCCTCCAACGAATAAATCAAGA
Amplifies part of the Vk5-2 variable gene:
(SEQ ID NO. 29)
Vk5-2F AAGTCCCCTGCATATCCACAAAA
(SEQ ID NO. 30)
Vk5-2R GCTGAGGCAATCCCACTGAGA

BAC clones are streaked on chloramphenicol plates to single colonies. Twelve single colonies are inoculated into 5 ml LB/20 μg/ml chloramphenicol each and grown overnight at 37° C. with shaking. BAC DNA from each culture is prepared and tested by PCR using the primers described above for the presence of specific markers for human immunoglobulin heavy chain loci as described below. The presence of at least 4 markers is verified to make sure that the BAC has not suffered any deletions. If the sequence of the BAC is known, then one can perform appropriate restriction enzyme digestions to look for diagnostic patterns of ethidium-stained bands on pulsed field gels. From 40 μl of DNA solution obtained from the 5 ml culture, 2-5 μl are digested and the fragments separated on a pulsed field gel of 1% agarose in 0.5× TBE and containing ethidium bromide running at 6 V/cm for 22 hours with reverse times of 0.1-1 second. BAC DNA is co-transfected into cES cells either as circular DNA or linearized. For linearized DNA, about 15 μg circular DNA are digested with NotI or other enzymes such as AscI or BsiWI that cut only in the vector backbone (and not in the insert) to release the vector backbone from the insert. The insert is purified away from the vector by electrophoresis on a pulsed field gel of 1% low melting point agarose/0.5× TBE/ethidium bromide under the same conditions as above. The agarose fragment containing the BAC insert DNA is excised and the agarose melted at 70° C. in the presence of 1×β-agarase I buffer (New England Biolabs). The agarose solution is equilibrated to 42° C. for 10 minutes and 1 μl β-agarase I (New England Biolabs) is added per 100 μl agarose volume and digested at 42° C. for 2 hours. The resulting mixture is heated to 70° C. for 15 minutes and then stored at 4° C. before transfection.

### Example 7

### Development of Antibody Diversity in the Transgenic Chicken

In contrast to humans, where Ig-negative precursor cells in the bone marrow continuously develop throughout life into cells expressing the primary antibody repertoire, in chickens such Ig-negative precursors exist only during development. In the mature chicken V(D)J recombination has ceased and the B cell compartment is maintained by proliferation of Ig-expressing cells in the periphery.

The Ig loci of chickens have been characterized in some detail. The chicken contains one heavy and one light chain locus per haploid genome; a second light chain locus has not been found. Both heavy and light chain genes rearrange V_{H}, D and J_{H} or V_{L} and J_{L} segments together into a functional variable region, by RAG-mediated recombination via signal sequences highly homologous to the mammalian versions. In chickens, the heavy and light chain genes rearrange at the same time, and there is no surrogate light chain, unlike in humans where the heavy chain rearranges first and combines with the VpreB/λ5 surrogate light chain. The chicken Ig gene segments that rearrange are the same as in humans, but their numbers are reduced down to the minimum: each locus contains only a single functional V gene (V_{H} or V_{L}) and single J segment (J_{H} or J_{L}). In addition, the heavy chain contains a cluster of 16 highly related D segments located between the single V and J segments. During the end joining process, no N nucleotides are added. Thus gene rearrangement of the germline chicken loci involves essentially all of the same cis- and trans-acting elements as in humans, but does not generate any appreciable combinatorial or junctional diversity because of the smaller numbers of V's, D's, J's and the lack of N nucleotide addition and may simply ensure allelic exclusion. Instead, accumulation of diversity begins after V(D)J rearrangement and is derived from gene conversion using upstream pseudogene sequences to mutate the expressed V. After productive rearrangement of the Ig loci and expression of a surface B cell receptor, the V region sequence starts to be mutated by replacement of short stretches of sequence derived from the upstream pseudogene pool. Ongoing gene conversion, at least in the bursa, produces an enormous number of different possible idiotypes, and also acts during affinity maturation in the germinal centers to produce antibodies of high affinity. Since the light chain pseudogene pool has been completely sequenced the source of templated mutations can be unequivocally assigned to specific pseudogenes. In addition to templated mutations, other mutations are often found that cannot be ascribed to any of the pseudogenes.

Chickens express immunoglobulins of three different classes: IgM, IgY and IgA. As in humans, the first Ig to be expressed during B cell development is surface IgM (sIgM), which interacts with an Igα/β heterodimer to induce downstream signaling and B cell progression (ref). Class switching to IgY and IgA production follows a secondary immune response. Juxtaposed to the heavy chain constant region genes lie switch repeats with sequence homology to mouse switch repeats, suggesting a highly conserved mechanism for class switch recombination and the expectation that switching of the human heavy chain will occur in the chicken. Chicken IgY is the functional equivalent of human IgG, but the heavy chain constant region has four Cν domains as opposed to three Cγ domains plus a hinge in IgG. Despite this apparent structural difference, experiments have shown that human IgG and IgA can adopt at least some of the normal functions of chicken Ig. Normally, maternal chicken IgY is scavenged from the blood and deposited in the egg yolk via a receptor-mediated process, (Mohammed, S M et al., “Deposition of genetically engineered human antibodies into the egg yolk of hens,” Immunotechnology, 1998 October; 4(2):115-25; Morrison, S L et al., “Sequences in antibody molecules important for receptor-mediated transport into the chicken egg yolk,” Mol Immunol. 2002 January; 38(8):619-25). In one set of experiments, cDNAs encoding humanized IgG and IgA monoclonal antibodies were transfected into chicken DT40 cells, an immortalized pre-B cell line, and stable cell lines expressing either hIgG or hIgA monoclonals were derived (Mohammed, S M et al., “Deposition of genetically engineered human antibodies into the egg yolk of hens,” Immunotechnology, 1998 October; 4(2):115-25; Morrison, S L et al.). The cells were then injected into chickens where they remained in circulation for many days, secreting human antibody into the blood. In the DT40 containing chickens, both hIgG and hIgA were also found in egg yolk, suggesting the human proteins share enough structural homology to chicken Ig to be recognized and transported into the yolk by the transport machinery. Eggs of a transgenic chicken expressing human immunoglobulins should thus be packed efficiently with human polyclonal Ig. (See Etches et al., entitled “Production of Proteins In Eggs,” PCT/CA98/00792).

To produce a diverse human polyclonal antibody repertoire, simplified constructs containing rearranged VJ and VDJ variable gene segments joined to light and heavy chain constant regions, respectively, are built and transfected into chicken ES cells. Transfected ES cells are differentiated in vivo into B cells by injection into early embryos. Transgenic B cells are analyzed for expression of human Ig mRNA by RT-PCR and by FACS for expression of human Ig protein on the surface of B cells. Progression of B cells through development is monitored using antibody staining to cell surface markers. Antibody diversity us then analyzed by sequencing of RT-PCR products. Human heavy chain transgenes enable class switching from IgM to IgG during an immune response and increased diversity is provided by inclusion of pseudogene sequences upstream of the light and heavy chain VJ or VDJ sequences.

### Example 8

### Human Kappa Light Chain Transgenesis

The kappa light chain BAC is extensively characterized by PCR for the presence of the predicted Ig gene elements, and by pulsed-field gel analysis for the predicted restriction map with rare cutters, using the human genome sequence as a reference. BAC libraries are retrofitted of BACs with selectable markers (Wang, Z., Engler, P., Longacre, A., Storb, U. (2001) An efficient method for high-fidelity BAC/PAC retrofitting with a selectable marker for mammalian cell transfection. Genome Research 11, 137-42) a CX-neo selectable marker that is known to confer G418 resistance to chicken ES cells was also inserted into the BAC in the vector backbone.

A large number of transfections with linearized kappa light chain BAC were performed. 8 neo^{R} clones were obtained, and 3 of these retained the BAC in its entirety, as judged by the presence of 6 markers distributed across the BAC. These three clones are injected into Stage X (ten) embryos to generate high grade somatic chimeras with extensive contribution to the feathers and hematopoietic system. Feather pigmentation is used because the host embryos are from a white strain of chickens (White Leghorn) and the ES cells are derived from a strain with black-striped feathers (Barred Plymouth Rock). Blood chimerism was measured by GFP fluorescence since all of the lines had a GFP gene previously transfected into them (integrated independently of the BAC). The numbers of GFP+ cells were determined by FACS analysis or by counting cells under the microsope.

Two sources of B lymphocytes are used: blood drawn from the wing vein of juveniles and adults, and embryonic bursa. Peripheral blood lymphocytes (PBL) are purified from whole blood by standard Ficoll gradient centrifugation. Since the PBL preparation is about 10-20% B lymphocytes and 80-90% T lymphocytes, staining with anti-Bu-1 antibodies in FACS analysis is used to verify the presence of B cells. Bursae from chicks collected just prior to hatch (at E19; hatch is at E21) provide pure B cells with little contamination from non-lymphoid cells, but only ˜10⁶ cells per bursa, and the animals must be euthanized.

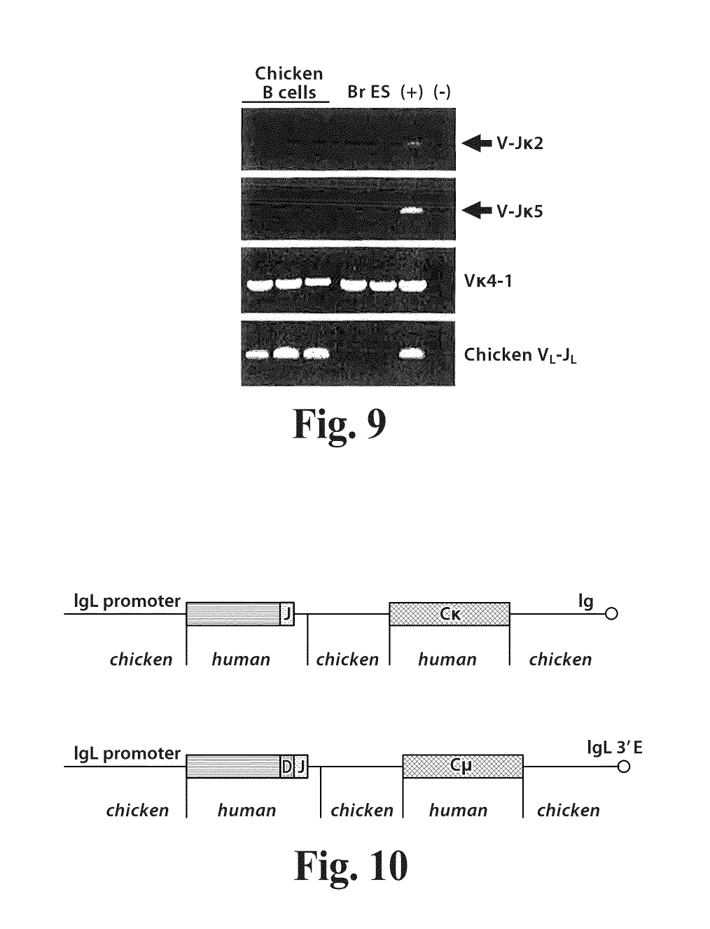

PCR assays for detection of Vκ-Jκ rearrangement were developed (FIG. 2). Forward primers that hybridize in each Vκ present on the BAC, and two reverse primers in the J region were designed to amplify all possible V-J combinations with PCR products under 1 kb to ensure robust amplification. Because the first two V's, Vκ1-5 and V□1-6, are highly conserved, only one primer was necessary to detect both; in total 6 sets of primers were used. A set of reactions to amplify rearrangements involving Vκ4-1 and Jκ2 or Jκ5 is shown in FIG. 9. Referring to FIG. 9, DNA samples from B cells, brain (Br), or ES cells (ES) containing the BAC were analyzed, as were positive controls (+) and no template (−) for each primer set. Top two panels, PCR primers to detect V-Jκ2 or V-Jκ5 gene rearrangements were used. Arrows point to the correct size band, seen in the control but not in chicken B cells. The faint band in brain is the wrong size. Other assays were performed for V-J1, V-J3 and V-J4 rearrangements (not shown). The positive control was human leukocyte DNA. Third panel, PCR to verify the presence of the BAC in the chimera samples. Positive control was human leukocyte DNA. Fourth panel, PCR for rearrangement of the endogenous chicken light chain, indicating the presence of B cells in the samples. Positive control was chicken B cell DNA.

To make sure that BAC-containing cells were present in the samples, a primer pair to detect non-rearranged alleles of Vκ4-1 is also used. To make sure the samples contained authentic B lymphocytes, an assay for rearrangement of the endogenous chicken light chain is also employed. Human leukocyte DNA (with a 5-10% B lymphocyte component) served as a positive control template for the human primers. For example, the most J-proximal V, Vκ4-1, is used at a frequency of about 9% in κ-expressing B cells. These positive controls estimate the sensitivity of the assays and 1 in 1000 cells with a rearranged allele of the BAC can be detected. Negative control templates were non-lymphoid tissues from chimeras, such as brain or muscle, or ES cells, which should contain only non-rearranged Ig alleles. In addition, primers were developed and optimized for RT-PCR detection of human Igκ cDNA (not shown), using human EST sequences as positive controls.

### Example 9

### Human/Chicken Hybrid Pre-Arranged Constructs

The production of a diverse repertoire of human polyclonal antibodies will depend on the ability of human Ig to perform all the critical functions that endogenous chicken Ig performs during B cell development: cell surface expression, participation in signal transduction mechanisms, and affinity maturation. Since gene rearrangement plays a limited role in chicken antibody production, “pre-rearranged” constructs can be created to resemble a normal, rearranged Ig locus, thereby signaling to the developing B cell to proceed in development. This construct design eliminates difficulties inherent in achieving VJ rearrangement of a human gene in the chicken.

In designing simplified constructs for expression of human Ig in chicken B cells, the principal elements are: a variable gene region (consisting of VJ or VDJ fused elements), a constant region gene, and transcriptional enhancer elements for expression in B cells. Upstream of the V segment must be a promoter containing an “octamer” sequence which is recognized by transcription factors, and a TATA box. To produce fully human sequence antibodies, the V, J and C coding sequences must be human, but to maximize the efficiency of the system, selected portions of the promoters, enhancers and intervening sequences are chicken sequences. For example, the most divergent human sequences may be replaced with their chicken counterparts and intervening chicken DNA is used to the extent possible. In particular, a 3′ enhancer element has been identified in the chicken locus that may be important for expression. Combining the chicken flanking sequences with the human coding sequences produces a functional Ig construct in the chicken.

For the kappa light chain, the human kappa light chain VJ element is PCR amplified from human genomic DNA, using one primer spanning the ATG initiator codon in the V leader exon and the other downstream of Jκ1. Commercially available human leukocyte genomic DNA, with a 5-10% B cell component, contains rearranged alleles of the Ig loci with fused VJ elements and VDJ elements. The PCR product contains a complete variable region coding sequence, ready to fuse to the chicken Ig light chain ATG, and the mRNA splice donor at the 3′ end of the J for splicing to the constant region exon. The kappa V of choice is from the Vκ1 family, the largest of the Vκ families, for reasons related to future gene conversion strategies involving the human transgenes. The PCR product is about 0.5 kb in size and is cloned and sequenced to make sure no deleterious mutations have been introduced during amplification and to make sure the variable region is from a productive gene rearrangement. The primer spanning the ATG introduces an NcoI site for ligation to the chicken promoter region at the NcoI site present at the endogenous chicken light chain initiator methionine codon. The transgene encodes a fully human Ig, but employs chicken transcriptional control sequences for expression in B cells. For the constant region, PCR is used to amplify the human Igκ constant region gene (a product of about 0.5 kb). To save time, all of these PCR reactions and their cloning and sequencing can proceed in parallel before final assembly of the construct.

For the human heavy chain transgene, the variable gene segments will be PCR amplified from human genomic DNA using a primer spanning the ATG in the V leader exon and another primer downstream of J_{H}1. The product obtained should contain a VDJ element and will be cloned and sequenced to make sure it is capable of encoding functional Ig.

Referring to FIG. 10, one human Vκ, 3 Jκ gene segments, and the Cκ constant region will be PCR amplified and assembled with chicken intervening sequences and flanking regions. (Intron/exon structure within the V and C Genes is not shown). The source of each part of the construct, human or chicken, is designated under the line. The recombination signal sequences are shown as triangles adjacent to V and J. The location of the chicken 3′ enhancer is shown.

The chicken genomic IgL flanking sequences are obtained from a 10.5 kb SacI fragment of the locus that has been characterized by Reynaud and colleagues (Reynaud, Claude-Agnes, Anquez, Viviane, Grimal, Helene, Weill, Jean-Claude (1987), “A Hyperconversion Mechanism Generates the Chicken Light Chain Preimmune Repertoire, Cell 48, 379-388). This fragment contains all of the sequences sufficient to express chicken light chain in transgenic mice. The regions taken from this construct are: the upstream flank and IgL promoter (as an EcoRI-NcoI fragment), the J-C intervening sequence (PCR amplified with BglII-HindIII sites at the ends), and the downstream flank (as a SalI fragment). The PCR products of the human gene segments are designed with the appropriate restriction sites at the ends for ligation to these chicken fragments. For example, the chicken upstream flank and promoter are joined to the human V sequence at the ATG initiator methionine codon via an NcoI site. At the 3′ end of the human, V an XmaI site is added for joining to the V-J intron. The final size of this construct is about 10 kb.

For stable transfection of these constructs into chicken ES cells, a neo^{R} selectable marker is included, driven by the CX promoter, a hybrid of the chicken β-actin promoter and CMV enhancer. Since the strong promoter-enhancer elements used to drive expression of the selectable marker may have an effect on transgene expression, the marker may be removed from ES cells before chimeric animals are made. To do so, the neo^{R} gene is flanked by loxP sites (“floxed”) and removed by Cre-mediated recombination. To screen for the excision reaction, a GFP gene is included adjacent to the neo in between the two loxP sites, and cells that have successfully looped out and lost the GFP-neo are no longer green fluorescent. An intact copy of the Ig transgenes is left behind and has no additional selectable markers or promoters/enhancers to interfere with B cell-specific expression.

There will be various modifications, improvements, and applications of the disclosed invention that will be apparent to those of skill in the art, and the present application encompasses such embodiments to the extent allowed by law. Although the present invention has been described in the context of certain preferred embodiments, the full scope of the invention is not so limited, but is in accord with the scope of the following claims. All references, patents, or other publications are specifically incorporated by reference herein. All references, patents, or other publications

1.-20. (canceled)

21. An isolated human polyclonal antibody obtained from a transgenic chicken in response to antigen stimulation, wherein the transgenic chicken has a functional disruption of an endogenous chicken immunoglobulin heavy chain and light chain gene and comprising a transgene comprising a nucleic acid molecule encoding a human immunoglobulin heavy chain and a human immunoglobulin light chain.

22. The antibody of claim 21, wherein the human immunoglobulin heavy chain is comprised of at least one human V gene, at least one human heavy chain D gene, and at least one human heavy chain J gene.

23. The antibody of claim 21, wherein the transgene comprises a plurality of human heavy chain V genes.

24. The antibody of claim 21, wherein the transgene further comprises a chicken B lymphocyte specific regulatory region operably linked to the human immunoglobulin heavy chain and the human immunoglobulin light chain.
